SEQUENCE LISTING (57 sequences)

SEQ ID NO: 1 - DNA, 120 bp, Artificial Sequence
  Description of Artificial Sequence Synthetic consensus sequence
acgatcgaacgggaatggataagaggcttgtgggattgacgtgatagggtagggttggctatactgctggtggcgaactccaggctaataatctgaagcgcatggatacaagttatcctt

SEQ ID NO: 2 - DNA, 120 bp, Zea mays
acgatcgaacgggaatggataggaggcttgtgggattgacgtgatagggtagggttggctatactgctggtggcgaactccaggctaataatctgaagcgcatggatacaagttatcctt

SEQ ID NO: 3 - DNA, 120 bp, Triticum aestivum
acgagcgaacgggaatggataagaggcttgtgggattgacgtgatagggtagggttggctatactgctggtggcgaactccaggctaataatctgaagcgcatggatacaagttatcctt

SEQ ID NO: 4 - DNA, 120 bp, Oryza sativa; identical to SEQ ID NO: 1
  Oryza sativa japonica

SEQ ID NO: 5 - DNA, 120 bp, Oryza sativa; identical to SEQ ID NO: 1
  Oryza sativa indica

SEQ ID NO: 6 - DNA, 118 bp, Nicotiana tabacum
gtcgttcaatgagaatggataagaggctcgtgggattgacgtgagggggcagggatggctatatttctgggagcgaactccgggcgaatatgaagcgcatggatacaagttatgcctt

SEQ ID NO: 7 - DNA, 120 bp, Marchantia polymorpha
tgttgttttcttgttattgaaagaggcttgtgggattgacataatagggtaggtatgggtatactagaaatgagcttcaagctaatatgaagtgaatgaaaaataaacataagttatcta

SEQ ID NO: 8 - DNA, 120 bp, Artificial Sequence
  Description of Artificial Sequence Synthetic consensus sequence
ggggaagggatataactcagcggtagagtgtcaccttgacgtggtggaagtcatcagttcgagcctgattatccctaaacccaatgtgagttttttctattttgacttactcccccgcca

SEQ ID NO: 9 - DNA, 120 bp, Zea mays
ggggaagggatataactcagcggtagagtgtcaccttgacgtggtggaagtcatcagttcgagcctgattatccctaaacctaatgtgagttttttctattttgacttactcccccacca

SEQ ID NO: 10 - DNA, 120 bp, Triticum aestivum; identical to SEQ ID NO: 8

SEQ ID NO: 11 - DNA, 120 bp, Oryza sativa
  Oryza sativa japonica
ggggaagggatataactcagcggtagagtgtcaccttgacgtggtggaagtcatcagttcgagcctgattatccctaaacccaatgtgagttttttctattttgacttactccccccgcc

SEQ ID NO: 12 - DNA, 120 bp, Oryza sativa; identical to SEQ ID NO: 11
  Oryza sativa indica

SEQ ID NO: 13 - DNA, 119 bp, Nicotiana tabacum
ggggaagggatataactcagcggtagagtgtcaccttgacgtggtggaagtcatcagttcgagcctgattatccctaagcccaatgtgagtttttctagttggatttgctcccccgccg

SEQ ID NO: 14 - DNA, 120 bp, Marchantia polymorpha
agaatagggatataactcagcggtagagtatcaccttgacgtggtggaagtcatcagttcgaacctgattatccctaaaaatagttaaagcattttgatttgttgttttcttgttattga

SEQ ID NO: 15 - DNA, 120 bp, Artificial Sequence
  Description of Artificial Sequence Synthetic consensus sequence
aaccctcttcaaatatggaacatggattctggcaatgcaacgtagttgggtcctcatatcgatccgaatgaatcattctttctacagaggtcaatctttgcctattaggcaagaggatag

SEQ ID NO: 16 - DNA, 120 bp, Zea mays
aacccgcttcaaatatggaacatggattctggcaatgcaacggagttgggtcctcatatcgatccgaatgaatcagtctttctacagaggtcaatctttgcctattaggcaagaggatag

SEQ ID NO: 17 - DNA, 120 bp, Triticum aestivum; identical to SEQ ID NO: 16

SEQ ID NO: 18 - DNA, 119 bp, Oryza sativa
  Oryza sativa japonica
aaccctcttcaaatatggaacatggattctgcaatgcaacgtagttgggtcctcatatcgatccgaatgaatcattctttctacagaggtcaatctttgcctattaggcaagaggatag

SEQ ID NO: 19 - DNA, 120 bp, Oryza sativa
  Oryza sativa indica
aaccctcttcaaatatggaacatggattctggcaatgcaacgtagttgggtcctcatatcgatccgaatgaatcagtctttctacagaggtcaatctttgcctattaggcaagaggatag

SEQ ID NO: 20 - DNA, 120 bp, Nicotiana tabacum
aggtcaacctctttcaaatatacaacatggattctggcaatgtagttggactctcatgtcgatccgaatgaatcatcctttccacggaggtaaatctttgcctgctaggcaagaggatag

SEQ ID NO: 21 - DNA, 120 bp, Artificial Sequence
  Description of Artificial Sequence Synthetic consensus sequence
tgtgagttctttcttccgtgatgaactgtcggcaccagtcctmcattttttctctgtggaccgaggagaaagggggctcagcaggaagaggattgtaccatgagagaagcacagaggtca

SEQ ID NO: 22 - DNA, 120 bp, Zea mays
tgtgagttctttcttccgtgatgaactgtcggcaccagtcctacattttttctctgtggaccgaggagaaagggggctcagcaggaagaggattgtaccatgagagaagcacagaggtca

SEQ ID NO: 23 - DNA, 121 bp, Triticum aestivum
tgtgagttctttcttccgtgatgaactgtcggcaccagtcctacatttttttctctgtggaccgaggagaaagggggctcagcaggaagaggattgtaccatgagagaagcacagaggtca

SEQ ID NO: 24 - DNA, 120 bp, Oryza sativa
  Oryza sativa japonica
tgtgagttctttcttccgtgatgaactgtcggcaccagtcctccattttttctctgtggaccgaggagaaagggggctcagcaggaagaggattgtaccatgagagaagcacagaggtca

SEQ ID NO: 25 - DNA, 120 bp, Oryza sativa; identical to SEQ ID NO: 24
  Oryza sativa indica

SEQ ID NO: 26 - DNA, 120 bp, Nicotiana tabacum
atcccgacttagtgagtctgatgaattgttggcaccagtcctacattttgtctctgtggaccgaggagaaaaggggctcggcgggaagaggagtgtaccatgagagaagcaaggaggtca

SEQ ID NO: 27 - DNA, 120 bp, Zea mays
tggtttaactgaataagtaaagaaaatgggccctttctcttcgtctcagatcgatggatcttctcgattggaagatctcccatatggataatacacattccagttgaccgagcctaatgc

SEQ ID NO: 28 - DNA, 120 bp, Zea mays
cctgagtactaactacatcaaacaatttgcattaatgaaactgtgtaaatccaccggataatcaaaaatacgcatgtctgatgaaatggttgttgctatctgtttccataacgaatcctt

SEQ ID NO: 29 - DNA, 120 bp, Artificial Sequence
  Description of Artificial Sequence Synthetic consensus sequence
gtcgatggctattcacagctactaccttaacaccaaagaagagttcgacccaatgctttatttctgtcttagtgaatcccgattcgacattaaaagtatattgattctttcccaataaac

SEQ ID NO: 30 - DNA, 120 bp, Zea mays; identical to SEQ ID NO: 29

SEQ ID NO: 31 - DNA, 120 bp, Triticum aestivum; identical to SEQ ID NO: 29

SEQ ID NO: 32 - DNA, 120 bp, Oryza sativa; identical to SEQ ID NO: 29
  Oryza sativa japonica

SEQ ID NO: 33 - DNA, 120 bp, Oryza sativa; identical to SEQ ID NO: 29
  Oryza sativa indica

SEQ ID NO: 34 - DNA, 120 bp, Nicotiana tabacum
gtcgatgactattcatagctattaccttgacaccaaagaagagttcgacccaatgctttatttctgtcctagttgatcctgattcgacattagaagtatattgatttttccccaataacc

SEQ ID NO: 35 - DNA, 120 bp, Artificial Sequence
  Description of Artificial Sequence Synthetic consensus sequence
ttagtttttttctctatctagaagtggaatagaataacccggttgaagggtaatgatcatacgtctgtaatgcattgtatgtcctagaatagggcccattcttctaccctttccgggtag

SEQ ID NO: 36 - DNA, 120 bp, Zea mays
ttagttcgtttctctatctagaagtggaatagaataacccggttgaagggtaatgatcatacgtctgtaatgcattgtatgtcccagaataggtcccattcttctaccctttccgggtag

SEQ ID NO: 37 - DNA, 120 bp, Triticum aestivum; identical to SEQ ID NO: 35

SEQ ID NO: 38 - DNA, 120 bp, Oryza sativa; identical to SEQ ID NO: 35
  Oryza sativa japonica

SEQ ID NO: 39 - DNA, 120 bp, Oryza sativa; identical to SEQ ID NO: 35
  Oryza sativa indica

SEQ ID NO: 40 - DNA, 119 bp, Nicotiana tabacum
taagttcttttctttctaagaggtggaatagaataacccggttgaagcgtaatgatcatacgtctgtaatgcattgtatgtcccataataggtcccattcttctactctttcccggaag

SEQ ID NO: 41 - DNA, 126 bp, Artificial Sequence
  Description of Artificial Sequence Synthetic consensus sequence
tcttttcaatttcgattatctatatattagttcgtttctatttctagatatctatttctatatattagtattagttagtagtactattctattagttagtatagagatatatctatatctatatat

SEQ ID NO: 42 - DNA, 120 bp, Zea mays
tcttttcgatttcgattatctatatattagttcgtttctatttctagatatctatttctatatatctatatattagtattaatatctatatattagtattagttatctatatattagtat

SEQ ID NO: 43 - DNA, 126 bp, Triticum aestivum
tcttttcaatttcgattatctatatattagttcatttctatttctagatatctatttctatatattagtattagttagtagtactattagttaccgatcccggctctgtgagttctttcttccgtg

SEQ ID NO: 44 - DNA, 126 bp, Oryza sativa
  Oryza sativa japonica
tcttttcaatttcgattatctatatattagttgtttctatttctagatatctatttctatatattagtattagttagtagtactattctattagttagcgatcccggctctgtgagttctttcttc

SEQ ID NO: 45 - DNA, 127 bp, Oryza sativa
  Oryza sativa indica
tcttttcaatttcgattatctatatattagttcgtttctatttctagatatctatttctatatattagtattagttagtagtactattctattagttagcgatcccggctctgtgagttctttcttc

SEQ ID NO: 46 - DNA, 121 bp, Marchantia polymorpha
tcctttattttgcattttttttttaactatcaaaaattttttctttctatataatatatatatatctatatattatatagatatctatataatatatagagatatatctatatctatatat

SEQ ID NO: 47 - DNA, 121 bp, Artificial Sequence
  Description of Artificial Sequence Synthetic consensus sequence
tgagtcgtccgagatcctttcgatgacctatgttgtgttgaagggatatctatatgatccgatcgattgcataagacccgcggtagcaatagaacggggaaagtatacagaaaagacagtt

SEQ ID NO: 48 - DNA, 120 bp, Zea mays
tgagtcgtccgagatcctttcgatgacctatgttgtgttgaagggatatctatatgatccgatcgattgcataagacccgcggtagcatagaacggggaaagtatacagaaaagacagtt

SEQ ID NO: 49 - DNA, 121 bp, Triticum aestivum
tgagtcgtccgagatcttttcgatgacctatgttgtgttgaagggatatctatatgatccgatcgattgcataagacccgcggtagcaatagaacggggaaagtatacagaaaagacagtt

SEQ ID NO: 50 - DNA, 121 bp, Oryza sativa; identical to SEQ ID NO: 47
  Oryza sativa japonica

SEQ ID NO: 51 - DNA, 121 bp, Oryza sativa; identical to SEQ ID NO: 47
  Oryza sativa indica

SEQ ID NO: 52 - DNA, 119 bp, Nicotiana tabacum
tgggtctccgagatcctttcgatgacctatgttgaagggatatctatctaatccgatcgattgcgtaaagcccgcggtagcaacggaaccggggaaagtatacagaaaagacagttctt

SEQ ID NO: 53 - DNA, 1279 bp, Zea mays
cctgattatccctaaacctaatgtgagttttttctattttgacttactcccccaccacgatcgaacgggaatggataggaggcttgtgggattgacgtgatagggtagggttggctatactgctggtggcgaactccaggctaataatctgaagcgcatggatacaagttatccttggaaggaaagacaattccgaatccgctttgtctacgaataaggaagctataagtaatgcaactatgaatctcatggagagttcgatcctggctcaggatgaacgctggcggcatgcttaacacatgcaagtcgaacgggaagtggtgtttccagtggcgaacgggtgagtaacgcgtaagaacctgcccttgggaggggaacaacaactggaaacggttgctaataccccgtaggctgaggagcaaaaggagaaatccgcccaaggaggggctcgcgtctgattagctagttggtgaggcaatagcttaccaaggcgatgatcagtagctggtccgagaggatgatcagccacactgggactgagacacggcccagactcctacgggaggcagcagtggggaattttccgcaatgggcgaaagcctgacggagcaatgccgcgtggaggtggaaggcctacgggtcgtcaacttcttttctcggagaagaaacaatgacggtatctgaggaataagcatcggctaactctgtgccagcagccgcggtaagacagaggatgcaagcgttatccggaatgattgggcgtaaagcgtctgtaggtggcttttcaagtccgccgtcaaatcccagggctcaaccctggacaggcggtggaaactaccaagctggagtacggtaggggcagagggaatttccggtggagcggtgaaatgcattgagatcggaaagaacaccaacggcgaaagcactctgctgggccgacactgacactgagagacgaaagctaggggagcaaatgggattagagaccccagtagtcctagccgtaaacgatggatactaggtgctgtgcgactcgacccgtgcagtgctgtagctaacgcgttaagtatcccgcctggggagtacgttcgcaagaatgaaactcaaaggaattgacgggggcccgcacaagcggtggagcatgtggtttaattcgatgcaaggcgaagaaccttaccagggcttgacatgccgcgaatcctcttgaaagagaggggtgccctcgggaacgcggacacaggtggtgcatggctgtcgtcagctcgtgccgtaaggtgtt

SEQ ID NO: 54 - DNA, 504 bp, Zea mays
gtattaatatctatatattagtattagttatctatatattagtattagttagtagtactattctattagttagcgatccccggctctgtgagttctttcttccgtgatgaactgtcggcaccagtcctacattttttctctgtggaccgaggagaaagggggctcagcaggaagaggattgtaccatgagagaagcacagaggtcaacccgcttcaaatatggaacatggattctggcaatgcaacggagttgggtcctcatatcgatccgaatgaatcagtctttctacagaggtcaatctttgcctattaggcaagaggatagcaagttcgaaattctgtctcggtaggacatggatttctattactatgaaattcataaattagttaatgggggggctaccattatcctttttcttgtatgtgttcctaagagaaggaatttgtccatttcatgtttcgaggtctcaaaaaaagggcgtggaaacagatagaaactctgga

SEQ ID NO: 55 - DNA, 543 bp, Zea mays
tctgtttccataacgaatccttggtttaactgaataagtaaagaaaatgggccctttctcttcgtctcagatcgatggatcttctcgattggaagatctcccatatggataatacacattccagttgaccgagcctaatgctaattgttttgttccgaagcaaagatatccgcggaggccggttcgttcgtcctattctgatattcaggaccaagaggtcctggattctctttcggataggccctgaaaggagaagagaagctgaaatgccaacggacctctgtctattctctaattcacccgatccgatagtacccgtttttggaacgtccagtgccaaagtcactgaatgggtaagtcaccaatccaatccctttgacaaatcgggtgtcatattagatatcatattctatatatatagaaatatcatagaatagacatatagaatttttggttgggaaattcgaatgaatcattgagtgaaaaaggagcaaagaatgacaaaagatgagactctactagtcttcactcttgtggtttcct

SEQ ID NO: 56 - DNA, 529 bp, Zea mays
taatgatcatacgtctgtaatgcattgtatgtcccagaataggtcccattcttctaccctttccgggtagtcgatggctattcacagctactaccttaacaccaaagaagagttcgacccaatgctttatttctgtcttagtgaatcccgattcgacattaaaagtatattgattctttcccaataaacgaagactcttttctgtaaatactgcgtatttgattccatccataaatcgactttcccccctatgctctgagttccagtatcgataagaattcgagttcttattgttcttatgttatggtatgaatataccataccaattcgttatgtatggatgatggatgagattccatggatagagagccaattccaatagaccgttcccgttcgcgtgcatccagcaggaattgaacccgcaaatttaccaattatgagttgggcgctttaaccattcagccatggatgcttaacagggatcatcgtacatcgtaaataaccaattttcatatagaaagacatatca

SEQ ID NO: 57 - DNA, 1960 bp, Zea mays
ctcctatattgggagttactctacgtattgcttgacgtaaaaccaatagtggatttgtttctgtcttttgttgaatctttttcacggctcgatagagaatttgataagccaatgatttttttccgtctttcataatacggttaaccaccatgttaactaatcgattacgaaaaattggatcggattttgcggttcttttttctgcagtacctcgacgtgacatgagcgtgaaagaggttcaagaatccgttttctttttataagggctaaaaacgaatcacttatttttttggctttttggccccatattgtagggtggatctcgaaagataggaaagatctccctccaagccgtacatacgactttcatcgaatacggctttccacagaattctatagggatctatgagatcgagtatggaattctgtttactcactttaaattgagtatccgtttccctccttttcccgctaggaccggaaatcctgtattttccatatccatacgatcgagtccttaggtttccgaaatagtgtaatggaaaaagaagtgcttcgaatcattgctatttgactcggacctgttctgaaaaagtcgaggtatttcgaattgtttgttgacacggacaaagtaagggaaaacctctgaaagaatttccatattgaccttggacatataagagttccgaatcgaatctctttagaaagaagatcttttgtctcatggtagcctgctccagtccccttacgaaactttcgttattgggttagccatacacttcacatgtttctagcgattcacatggcatcatcaaatgatacaagtcttggataagaatctacaacgcactagaacgcccttgttgacgattctttactgcgacagcatctagggttcctcgaataatgcgatatctcacaccgggtaaatccttaacccttcctcctcttactaatactacagaatgttcttgtaaattatggccaataccaggtatataagcagtgatttcaaatccagaggttaatcgtactctggcaactttacgtaaggcagagttgggttttttggggttgatagtggaaaagtcgacagataagtcatccttactgtccctctacagaaccgtacatgagattttcacctcatacggctcctcgttcaattctttcgaagggatccttttcctcgttcgagagtctccgcccttcttccactccgtcccgaagactaactaagaccaattgagtcacgttttcatgttctaattgaacactttccatttatgattaaaggagaagattgttcttttaccaaacatatgcggatcaaatcacgtcttataataagaagaaatctttctcggtatcaatccccttgcccctcattctttgagaatcagaaggatccttttcgagtttccatttcttcattttgaatctgggctcttctatcttcgacttatttttttggctttattctttatttatttcatttcgatttttccctcttcctctatccctatcctctgaggtacagcgtttgcatcaatagagaactttttcctctgtatgaatcgatattattccaatttcttcccgaaacttcccaagaaaaatcccgaattggatccaaaattgacgggttaatgtgagcttatccatgcggttaggcactcttcaaataggaatccattttctaactggctttcgtgctttggtgagtcgtccgagatcctttcgatgacctatgttgtgttgaagggatatctatatgatccgatcgattgcataagacccgcggtagcatagaacggggaaagtatacagaaaagacagttcttttcgatttcgattatctatatattagttcgtttctatttctagatatctatttctatatatctatatattagtattaatatctatatattagtattagttatctatatattagtat

(19) United States
(12) Patent Application Publication
(10) Pub. No.: US 2015/0267214 A1
(43) Pub. Date: Sep. 24, 2015

(54) PLASTID TRANSFORMATION USING LINEAR DNA VECTORS

(71) Applicant: Arnold Jay Bendich, Seattle, WA (US)

(72) Inventors: Arnold Jay Bendich, Seattle, WA (US); Delene June Oldenburg, Olympia, WA (US)

(21) Appl. No.: 14/437,080

(22) PCT Filed: Mar. 13, 2013
(86) PCT No.: PCT/US2013/030775

§ 371 Date: Apr. 20, 2015

Related U.S. Application Data
(60) Provisional application No. 61/718,095, filed on Oct. 24, 2012.

(51) Int. Cl. C12N 15/82

(52) U.S. Cl. CPC C12N 15/8214; C12N 15/8207

(57) ABSTRACT

The present disclosure provides methods of plastid transformation using linear DNA vectors and plant tissues having substantially non-degraded plastid DNA. Also provided are linear DNA vectors useful for the methods provided herein, transplastomic plants or plant parts obtained by the methods provided herein, and progenies of such transplastomic plants or plant parts.

### CROSS REFERENCE TO RELATED APPLICATIONS

This application claims benefit under 35 U.S.C. §119(e) of U.S. Provisional Application No. 61/718,095 filed Oct. 24, 2012, the contents of which are incorporated herein by reference in their entirety.

### STATEMENT OF GOVERNMENT INTEREST

This invention was made with government support under Contract No. 2008-39211-19557 awarded by the US Department of Agriculture. The government has certain rights in this invention.

### SEQUENCE LISTING

The instant application contains a Sequence Listing which has been submitted in ASCII format via EFS-Web and is hereby incorporated by reference in its entirety. Said ASCII copy, created on Mar. 12, 2013, is named 034186-077150-PCT_SL.txt and is 20,932 bytes in size.

### TECHNICAL FIELD

The present disclosure relates to methods of plastid transformation, transplastomic plants and plant parts generated using such methods, and progenies of such transplastomic plants and plant parts.

### DESCRIPTION OF THE RELATED ART

Plastid transformation, as opposed to nuclear transformation, has numerous advantages. It allows high transgene expression levels, multi-gene engineering in a single transformation event, and transgene containment via maternal inheritance. In addition, plastid transformation lacks pleiotropic effects due to subcellular compartmentalization of toxic transgene products. Furthermore, this technique avoids other problems associated with nuclear transgene expression, such as gene silencing and position effects due the site of transgene integration. Despite its advantages, plastid transformation has been limited to certain dicots. Monocots such as maize, wheat, and rice have proved recalcitrant to this technique.

### SUMMARY

The methods and compositions described herein relate to vectors and transformation protocols that provide for high efficiency plastid transformation in any plant. In some embodiments, the plant can be a monocot, which have historically been recalcitrant to plastid transformation.

In one aspect, the present disclosure provides a method of plastid transformation, comprising: introducing a linear DNA vector into a plastid of a plant tissue, wherein (i) the plastid comprises substantially non-degraded plastid DNA, and (ii) the linear DNA vector comprises (1) a plastid DNA targeting sequence, and (2) a transgene of interest.

In certain embodiments, the plant is a cereal crop.

The transgene of interest may be selected from the group consisting of genes encoding therapeutic or prophylactic polypeptides, genes that provide or enhance herbicide resistance, insect resistance, fungal resistance, bacterial resistance, and stress tolerance, and genes that improve nitrogen fixation, mineral nutrition, plant yield, starch accumulation, fatty acid accumulation, protein accumulation, and photosynthesis.

In certain embodiments, the linear DNA vector further comprises a gene encoding a selection marker, such as a gene providing resistance against spectinomycin, streptomycin, kanamycin, hygromycin, chloramphenicol, glyphosate or bialaphos, a gene providing metabolism of mannose, or a gene encoding a fluorescent protein.

In certain embodiments, the plastid DNA targeting sequence comprises a terminal sequence of a plastid chromosomal DNA molecule. For example, the plastid terminal sequence is at least 90% identical to a portion of SEQ ID NO:1, 8, 15, 21, 29, 35, 41, or 47, which portion is at least 30 nucleotides in length.

In certain embodiments, the plastid terminal sequence comprises: at least 30 consecutive nucleotides of SEQ ID NO:2, 9, 16, 22, 27, 28, 30, 36, 42, 48, 53, 54, 55, 56, or 57 when the plant tissue is a maize tissue, at least 30 consecutive nucleotides of SEQ ID NO:3, 10, 17, 23, 31, 37, 43, or 49 when the plant tissue is a wheat tissue, at least 30 consecutive nucleotides of SEQ ID NO:4, 5, 11, 12, 18, 19, 24, 25, 32, 33, 38, 39, 44, 45, 50, or 51 when the plant tissue is a rice tissue, at least 30 consecutive nucleotides of SEQ ID NO:6, 13, 20, 26, 34, 40, or 52 when the plant tissue is a tobacco tissue, or at least 30 consecutive nucleotides of SEQ ID NO:7 or 46 when the plant tissue is a liverwort tissue.

In some embodiments, the linear DNA vector comprises a single-stranded overhang at either the 5′ end or the 3′ end, a single-stranded loop that may or may not covalently join the two DNA strands of the linear DNA vector, or a molecule that is not a nucleotide covalently joined to either the 5′ end or the 3′ end.

In some embodiments, the plant tissue is a non-green tissue, such as a portion of a mature embryo, a portion of a dark-grown seedling, a seed, or a portion of a seed.

In certain embodiments, the plastid is a proplastid, etioplast, or other non-green plastid.

In certain embodiments, step (a) is performed via biolistic bombardment of the plant tissue with microparticles coated with the linear DNA vector.

In certain embodiments, the method of plastid transformation further comprises (b) culturing the plant tissue from step (a) without light.

In some embodiments, the method of plastid transformation further comprises (c) regenerating a transplastomic plant from the plant tissue from step (a) or step (b).

In certain embodiments, the transplastomic plant is homoplasmic.

In another aspect, the present disclosure provides a transplastomic plant or a plant part obtained by the method disclosed herein.

In a related aspect, the present disclosure provides a progeny of a plant or plant part of a transplastomic plant obtained by the method disclosed herein.

In a further aspect, the present disclosure provides a linear DNA vector for plastid transformation in a plant, comprising: (1) a plastid DNA targeting sequence that comprises a plastid terminal sequence, and (2) an expression cassette that comprises: (a) optionally a promoter active in the plastids of the plant to be transformed, (b) a DNA insertion site for receiving a transgene of interest, (c) optionally one or more selection markers, and (d) optionally a DNA sequence encoding a transcription termination region active in the plastids of the plant to be transformed.

In certain embodiments, the linear DNA vector further comprises a transgene of interest inserted at the DNA insertion site.

### BRIEF DESCRIPTION OF THE DRAWINGS

FIGS. 1A-1F depict liverwort plastid transformation vectors and transgene integration. FIG. 1A depicts a schematic of vector pCS31. FIG. 1B depicts a schematic of vector LpCS31 linearized by SacI digestion. FIG. 1C depicts a schematic of vector TCpCS31 linearized by NotI/SacII digestion. The aadA/RBS fragment gives positive transformants. FIG. 1D depicts a schematic of integration into plastid genome by homologous recombination. FIG. 1E depicts a schematic of integration by end joining FIG. 1F depicts a schematic of integration by strand invasion. LBS: left border sequence, RBS: right border sequence, aadA: transgene with PEP promoter, 5′-UTR and 3′-UTR, P1, P4-P6: PCR primers flanking the border sequence, P2, P3: PCR primers within the transgene cassette, aL, aR: PCR primers for the aadA transgene.

FIGS. 2A-2C depict vectors for tobacco plastid transformation. FIG. 2A depicts a schematic of circular vector pPRV111A containing tobacco ptDNA LBS and RBS, aadA gene for spectinomycin selection flanked by tobacco psbA promoter/psbA 5′-UTR and psbA 3′-UTR. A polylinker with restriction sites is located between the LBS and promoter regions. The transgene cassette is accession U12812. Arrows indicate location of 16S rRNA and trnV genes. FIG. 2B depicts a schematic of linear vector LpPRV111A, generated by EcoRV digestion of pPRV111A. FIG. 2C depicts a schematic of linear vector TCpPRV111A, generated by SacI digestion of pPRV111A.

FIGS. 3A-3G depict vectors for maize plastid transformation. FIGS. 3A and 3C depict schematics of circular vectors pZMCP150 and pZMCP152 containing maize ptDNA LBS and RBS and the gfp gene flanked by maize 16S rRNA promoter/psbA 5′-UTR and psbA 3′-UTR. Arrows indicate location of 16S rRNA, ORF85, and trnV genes, and PCR primers. In RBS, End5 corresponds to the 5′ and 3′ ends of IRb, pZMCP150 and pZMCP152, respectively. FIGS. 3B and 3D depict schematics of linear vectors TCpZMCP150 and TCpZMCP152, generated by PstI/PvuII/SfiI digestion. The PvuII enzyme was included to reduce the chance of recircularization by linear vector fragments. Three other linear vectors were generated by restriction digestion: FIG. 3E depicts a schematic of TC2pZMCP152, which includes entire LBS with End5 and minimal RBS (27 bp) regions; FIG. 3F depicts a schematic of TC3pZMCP152, which includes entire RBS and minimal LBS (172 bp) without End5; FIG. 3G depicts a schematic of TC4pZMCP152, which includes entire LBS and RBS regions, but the end sequences are comprised of 113 and 205 bp from the cloning plasmid.

FIGS. 4A-4B depict images of maize embryonic tissue bombarded with plastid transformation vector. A single piece of callus derived from mature embryo following particle bombardment with circular vector pZMCP150 was imaged: for each pair of images white light is on left and GFP is on right. Autofluorescence of plant cell walls can be seen in both 4A and 4B. FIG. 4A depicts a region of tissue without GFP expression in plastids. FIG. 4B depicts a region of tissue with GFP expression in plastids.

FIGS. 5A-5D depict images of maize embryonic tissue bombarded with plastid transformation vectors. A single piece of callus derived from mature embryo following particle bombardment was imaged: for each pair of images white light is on left and GFP is on right. FIG. 5A depicts tissue with no DNA, FIG. 5B depicts tissue with linear vector TCpZMCP150, FIG. 5C depicts tissue with circular vector pZMCP152, and FIG. 5D depicts tissue with linear vector TCpZMCP152. Autofluorescence of plant cell walls can be seen with GFP filter.

FIGS. 6A-6B depict SEQ ID NOS: 1-14; ptDNA sequence alignment of portions of maize End5 (SEQ ID NOS:2 and 9) with ptDNA from wheat (SEQ ID NOS:3 and 10), rice (SEQ ID NOS:4, 5, 11, and 12), tobacco (SEQ ID NOS:6 and 13), and liverwort (SEQ ID NO:7 and 14). The consensus sequence is set forth in SEQ ID NOS:1 and 8. The terminal sequence of linear ptDNA, designated here and in FIG. 3 as End5 and as End1/5 in Table 6, was assessed for homology in five sequenced plastid genomes: wheat (Ta, Triticum aestivum, NC_{—}02782), rice (Osj, Oryza sativa japonica, X15901; Osi, Oryza sativa indica, AY522329), tobacco (Nt, Nicotiana tabacum, NC_{—}00189), and liverwort (Mp, Marchantia polymorpha, X04665). The entire End1/5 sequence alignment spans a 1278 bp region corresponding to nt 94920:96198 (FIG. 11) of the maize plastid genome, X86563. The data best fit an end located at nt 96976±60 in IRb and 127767±60 in IRa (see Table 6). FIG. 6A depicts a comparison of 120 bp of the 5′ end of End5 sequence at nt 94976 to nt 95095 to other plant ptDNAs. FIG. 6B depicts a comparison of 120 bp of the 3′ end of End5 sequence at nt 94857 to nt 94976 to other plant ptDNAs. Dots represent identical nucleotides (A/G/C/T) among each plant, capitalized letters indicate base changes within highly homologous regions, and lower case letters indicate non-homology outside of the highly homologous region

FIGS. 7A-7B depict SEQ ID NOS: 15-26; ptDNA sequence alignment of a portion of maize End2 (SEQ ID NOS:16 and 22) with the same plant ptDNAs as in FIG. 6. FIG. 7A depicts a comparison of 120 bp of the 5′ end of End2 sequence at nt 94143 to nt 94262 to other plant ptDNAs. FIG. 7B depicts a comparison of 120 bp of the 3′ end of End2 sequence at nt 94924 to nt 94143 to other plant ptDNAs. No analogous sequence was found for Mp.

FIGS. 8A-8B depict SEQ ID NOS: 27 and 28; a portion of 5′ end sequence of maize End3 (SEQ ID NO:27 and 28) was compared with the same plant ptDNAs as in FIG. 6, although no analogous sequences were found for any of the other five ptDNA sequences. FIG. 8A depicts 120 bp of the 5′ end of End3 sequence, nt 87402 to nt 87521. FIG. 8B depicts 120 bp of the 3′ end of End3 sequence, nt 87283 to nt 87402.

FIGS. 9A-9B. SEQ ID NOS: 29-40; ptDNA sequence alignment of portions of maize End4 (SEQ ID NO:30 and 36) with the same plant ptDNAs as in FIGS. 6A-6B. FIG. 9A depicts a comparison of 120 bp of the 5′ end of End4 sequence at nt 84555 to nt 84674 to other plant ptDNAs. FIG. 9B depicts a comparison of 120 bp of the 3′ end of End4 sequence at nt 84436 to nt 84555 to other plant ptDNAs. No analogous sequence was found for Mp.

FIGS. 10A-10B depict SEQ ID NOS: 41-52; ptDNA sequence alignment of a portion of maize End6 (SEQ ID NOS: 42 and 48) with the same plant ptDNAs as in FIG. 6A-6B. FIG. 10A depicts a comparison of 120 bp of the 5′ end of End6 sequence at nt 93863 to nt 93982 to other plant ptDNAs. No analogous sequence was found for Nt. FIG. 10B depicts a comparison of 120 bp of the 3′ end of End6 sequence at nt 93744 to nt 93863 to other plant ptDNAs. No analogous sequence was found for Mp.

FIG. 11 depicts SEQ ID NO: 53; the entire End1/5 sequence as determined from ClustalW alignments using sequencing data. The “true end” was assigned to the first nucleotide where the greatest degree of overlap was found among all individual sequencing outputs.

FIG. 12 depicts SEQ ID NO: 54; the entire End2 sequence as determined from ClustalW alignments using sequencing data. The “true end” was assigned to the first nucleotide where the greatest degree of overlap was found among all individual sequencing outputs.

FIG. 13 depicts SEQ ID NO: 55; the entire End3 sequence as determined from ClustalW alignments using sequencing data. The “true end” was assigned to the first nucleotide where the greatest degree of overlap was found among all individual sequencing outputs.

FIG. 14 depicts SEQ ID NO: 56; the entire End4 sequence as determined from ClustalW alignments using sequencing data. The “true end” was assigned to the first nucleotide where the greatest degree of was found among all individual sequencing outputs.

FIG. 15 depicts SEQ ID NO: 57; the entire End6 sequence as determined from ClustalW alignments using sequencing data. The “true end” was assigned to the first nucleotide where the greatest degree of overlap was found among all individual sequencing outputs.

### DETAILED DESCRIPTION

The present disclosure provides methods of plastid transformation, linear DNA vectors useful for such methods, transplastomic plants or plant parts obtained by such methods, and progenies of these plants and plant parts.

Standard methods of plastid transformation use circular vectors with transgene integration into the plastid genome occurring by homologous recombination using green plant tissues grown under light. Success in plastid transformation using such methods was observed only in certain dicot species, but not in cereals (monocots) such as maize, wheat and rice.

The methods of the present disclosure use novel combinations of linear DNA vectors and plant tissues that comprise substantially non-degraded plastid DNA (e.g., non-green tissues). Such methods allow successful plastid transformation in cereals and provide higher plastid transformation rates than those using circular DNA vectors and/or green plant tissues. The methods provided herein may generate transplastomic plants that are homoplasmic.

In one aspect, the present disclosure provides a method of plastid transformation that comprises introducing a linear DNA vector into a plastid of a plant tissue. The plastid to which the linear DNA vector is introduced comprises substantially non-degraded plastid DNA, and the linear DNA vector comprises a plastid DNA targeting sequence and a transgene of interest.

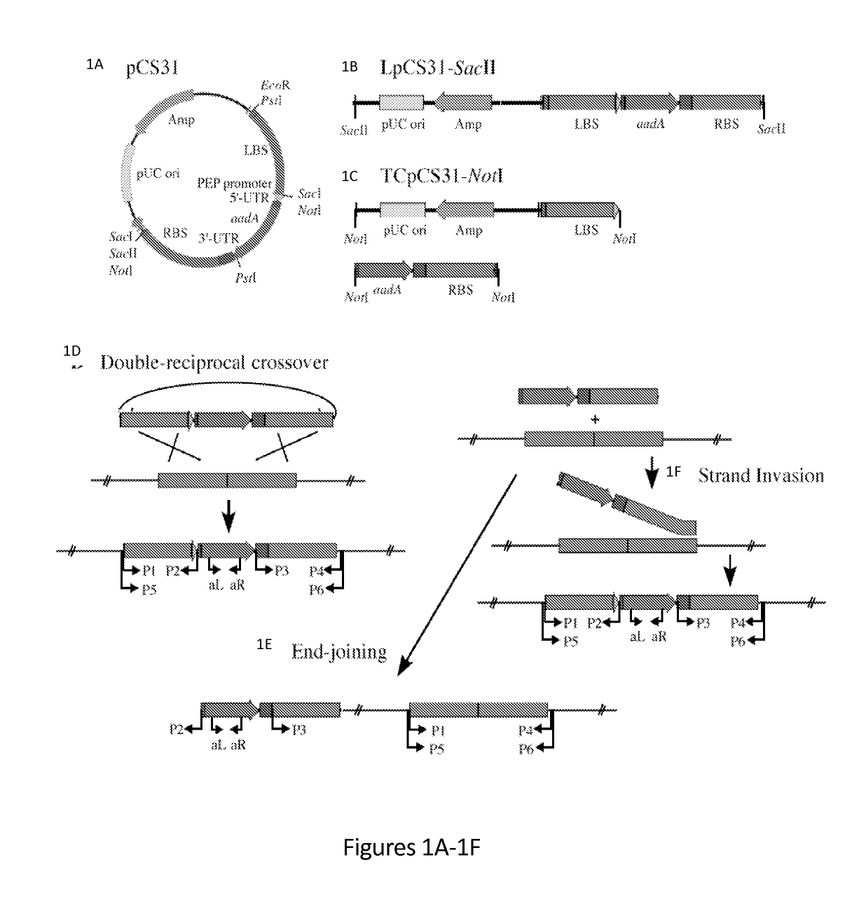

Traditionally, plastid transformation was accomplished by inserting a transgene carried on a circular DNA vector into resident plastid DNA (assumed to be exclusively in circular DNA molecules) by double-reciprocal recombination: crossovers both upstream and downstream of the transgene. It was also believed that recombination was extremely frequent within plastids and responsible for the two equimolar isomers of the circular chromosomal DNA (Palmer, Nature 301:92-3, 1983). Without being bound by theory, the present inventors hypothesize that using linear DNA vectors instead of circular DNA vectors could achieve higher plastid transformation rates via end-mediated incorporation of transgenes, such as end-joining and strand invasion (see, Example 1, FIG. 1 and Table 3). The present inventors further hypothesize that because most plastid DNA is actually linear, traditional circular transformation vectors do not resemble the resident plastid chromosome and would not be recognized as a “natural” chromosome. Instead, linear DNA vectors modeled after the endogenous linear ptDNA may be integrated into the plastid genome by the endogenous ptDNA replication machinery more efficiently than a circular vector.

In some embodiments described herein, a linear DNA molecule can permit the transfer of a transgene to a plastid. As used herein, a “linear DNA vector” refers to a DNA vector which, when present in a double-stranded form, has two strands, each having a 5′ and 3′ end which are not ligated to each other. In some embodiments of any of the aspects described herein, the DNA vector can be a non-circular DNA vector. As used herein, a “circular DNA vector” refers to a DNA vector which, when present in a double-stranded form, has two strands wherein each strand is a continuous loop with no physically identifiable 5′ or 3′ end. In some embodiments, a “circular DNA vector” can comprise a nicked circular DNA vector, e.g. a circular DNA vector with a first unbroken circular strand and a second strand with a 5′ and a 3′ end which are not ligated to each other. A linear DNA vector can include, by way of non-limiting example, a double-stranded DNA molecule wherein each strand has a free 5′ and 3′ end; a double-stranded DNA molecule wherein the 3′ end of one strand is ligated to the 5′ end of the second strand (e.g. forming a single-stranded hairpin molecule); and other forms as described below herein. In some embodiments, a linear DNA vector can be obtained by linearizing a circular DNA vector using one or more restriction enzymes. In certain embodiments, a linear DNA vector comprises a replication origin functional in a host cell (e.g., bacteria and yeast). In certain other embodiments, a linear DNA vector does not comprise any replication origin functional in a host cell.

In certain embodiments, a linear DNA vector useful in plastid transformation comprises a plastid DNA-targeting sequence to facilitate integration of the linear DNA vector or a portion thereof into plastid DNA. In some embodiments, the plastid-targeting sequence can be located on one flank of the transgene sequence. In some embodiments, plastid-targeting sequences can be located on both flanks of the transgene sequence.

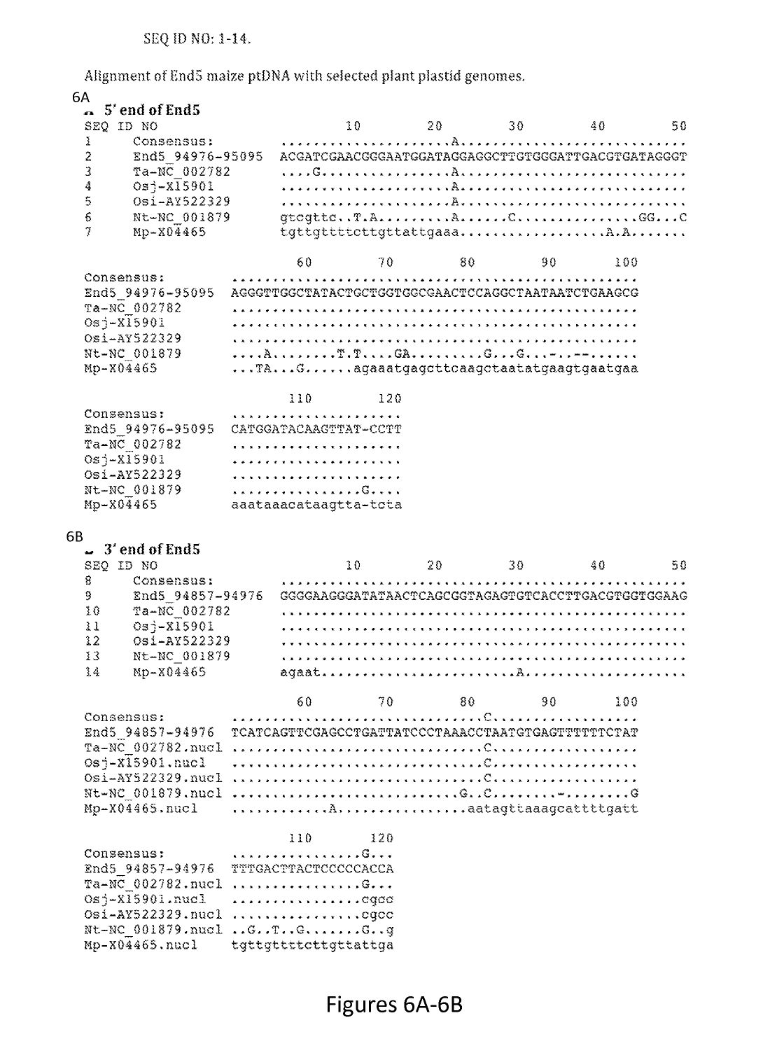

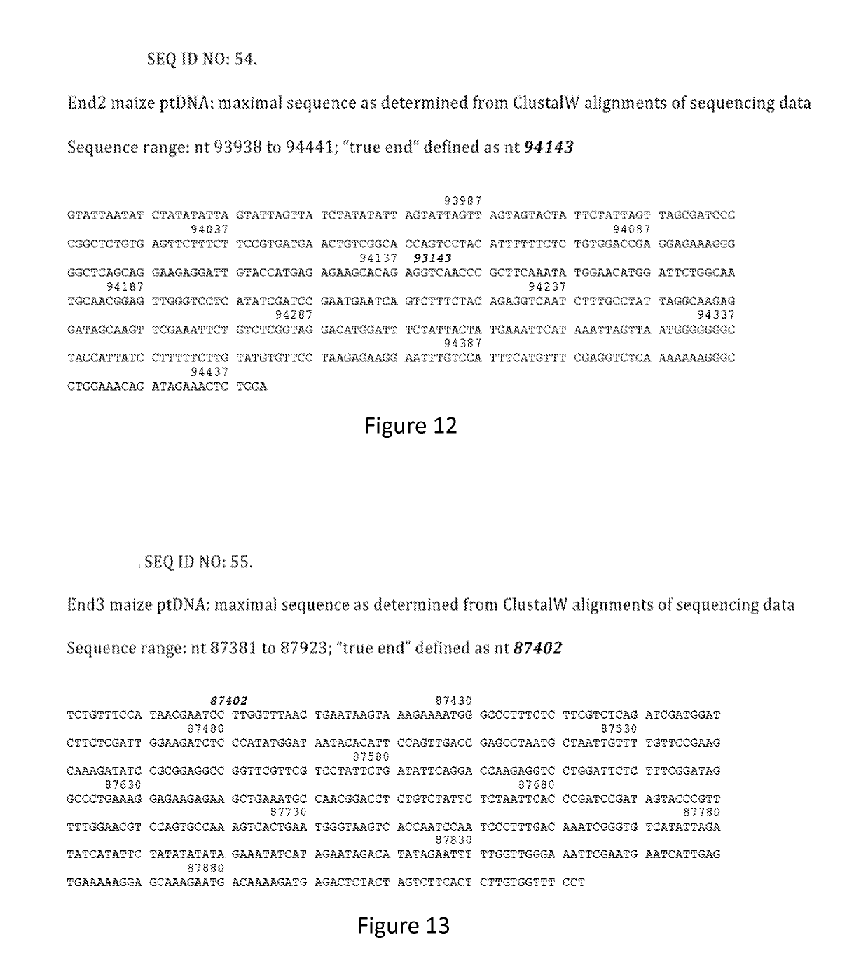

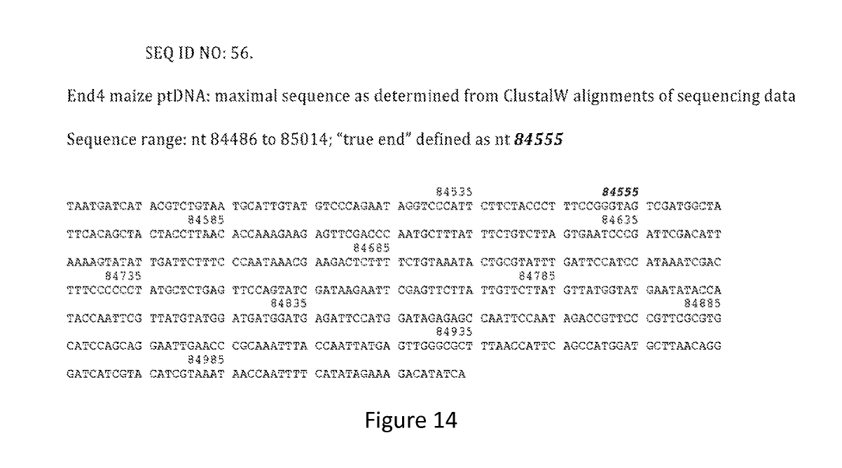

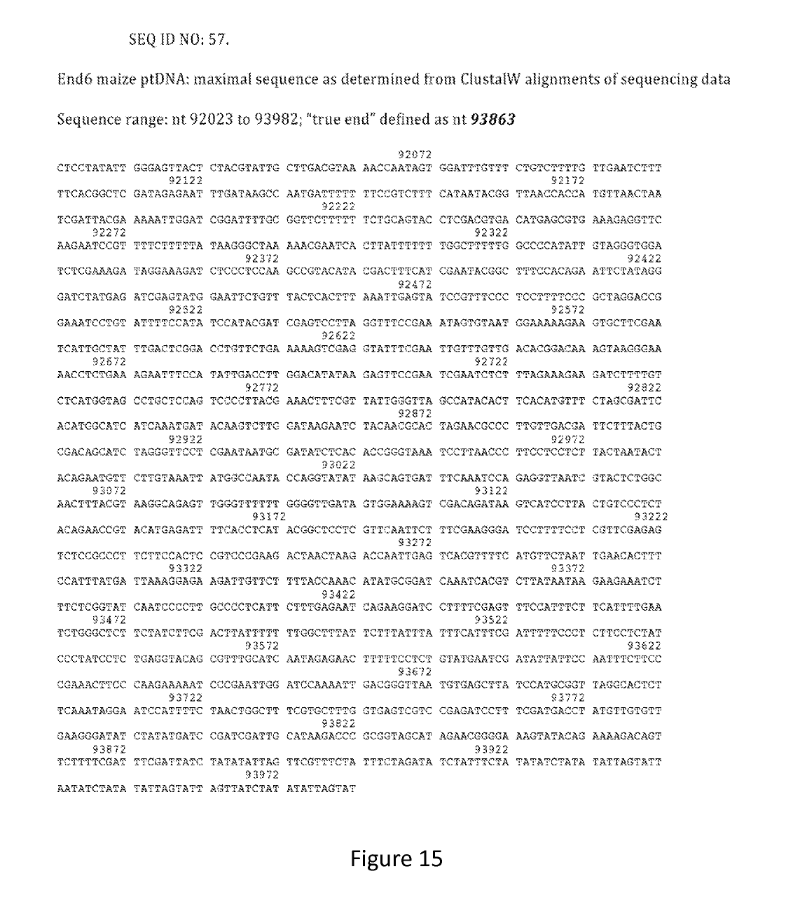

The plastid DNA-targeting sequence may be a sequence sufficiently similar to a sequence in the plastid genome of interest to allow homologous recombination between the plastid DNA-targeting sequence and the plastid genome. Any sequence sufficiently similar to a sequence in the plastid genome of interest may be included in the linear DNA vector. Exemplary plastid DNA-targeting sequences include the left border sequence (LBS) and the right border sequence (RBS) as provided in the Examples. Other preferred DNA-targeting sequences are sequences that include all or a portion of the End sequences as determined by the inventors including those described in Table 6 and FIGS. 6-15 and those that are composed of plastid expression control regions (such as promoters and terminators) that flank the transgene without additional LBS/RBS sequences. In certain embodiments, a linear DNA vector comprises two different plastid DNA-targeting sequences: one on each side of a transgene of interest also present in the linear DNA vector. In some embodiments, the presence of both plastid DNA-targeting sequences allows integration of the transgene via double-reciprocal homologous recombination, whereas the presence of a plastid DNA-targeting sequence at the end of the DNA vector permits end-mediated incorporation of the vector DNA into the plastid genome.

Without wishing to be bound by theory, the present inventors hypothesize that plastid end sequences are likely important for genome amplification during plastid DNA replication, and efficient plastid transformation may be achieved by using vectors comprising targeting sequences substantially identical or homologous to at least a portion of these ends. Thus, in certain embodiments, the plastid DNA-targeting sequence comprises a plastid terminal sequence that is identical to or homologous to a portion of a plastid end sequence. A “plastid end sequence” refers to a region of up to about 1000 bp in length (e.g. at least about 100 bp in length, at least about 200 bp in length, at least about 300 bp in length, at least about 400 bp in length, at least about 500 bp in length, at least about 600 bp in length, at least about 700 bp in length, at least about 800 bp in length, at least about 900 bp in length, or up to about 1000 bp in length, or e.g., from 100 to 1000 bp in length) (sequence determined as described in Example 3), adjacent to an end of a naturally-occurring linear plastid chromosome, either downstream of the 5′ end or upstream of the 3′ end. A “5′ end sequence” of a plastid DNA end refers to a region adjacent to the plastid DNA end located downstream of the 5′ plastid DNA end. A “3′ end sequence” of a plastid DNA end refers to a region adjacent to the plastid DNA end located upstream of the 3′ plastid DNA end.

Plastid end sequences are readily determined by one skilled in the art, e.g. plastid DNA sequences are known in the art for a number of species. Plastid DNA sequences are publicly available in databases for a number of plant species, e.g. in the Chloroplast DB, freely available on the world wide web at http://chloroplast.cbio.psu.edu/organism.cgi. Plastid DNA sequences are usually depicted as a “circular map” with the first nucleotide of the long single copy (LSC) region adjacent and connected to the last nucleotide of one of the inverted repeats (IRa). Nucleotide number 1 (nt=1) is designated as the first nucleotide of the LSC (or its equivalent for plastid DNA sequences that lack inverted repeats). The first and last numbered nucleotides of plastid DNA sequences do not, however, correspond to true ends of linear plastid DNA molecules. Determining the plastid end sequence of a given species or variety requires preparation of structurally intact plastid DNA and sequencing from the ends is described, e.g. in Example 3 herein.

In some embodiments, plastid end sequences can also include sequences homologous to any of the plastid end sequences disclosed herein. Homologs of any given nucleic acid sequence can be found, e.g., by using BLAST programs, e.g. by searching freely available databases of sequence for homologous sequences, or by querying those databases for annotations indicating a homolog. Such databases can be found, e.g. on the world wide web at http://ncbi.nlm.nih.gov/. In some embodiments, a homolog of a plastid end sequence, a plastid-targeting sequence, or a plastid terminal sequence is a sequence having at least 85% identity to a portion of a sequence described herein having a length of at least 25 bp, e.g. 85% or greater identity, 90% or greater identity, or 95% or greater identity over a portion of a sequence having a length of at least 25 bp.

As used herein, the term “plastid terminal sequence” refers to a sequence which is at least 85% identical to a portion of a plastid end sequence which is at least about 25 bp in length.

In some embodiments, a plastid terminal sequence is identical to a portion of a plastid end sequence that is 25-50, 50-100, 100-150, or 150-200 nucleotides in length. In certain embodiments, a plastid terminal sequence is identical to a portion of a plastid end sequence that is at least 25, 30, 35, 40, 45, or 50 nucleotides in length.

In certain embodiments, a plastid terminal sequence is at least 85%-90%, 90%-95%, or 95%-99% identical to a portion of a plastid end sequence, which portion may be 25-50, 50-100, 100-150, or 150-200 nucleotides in length. In some embodiments, a plastid terminal sequence is at least 85%-90%, 90%-95%, or 95%-99% identical to a portion of a plastid end sequence, which portion may be at least 25, 30, 35, 40, 45, or 50 nucleotides in length.

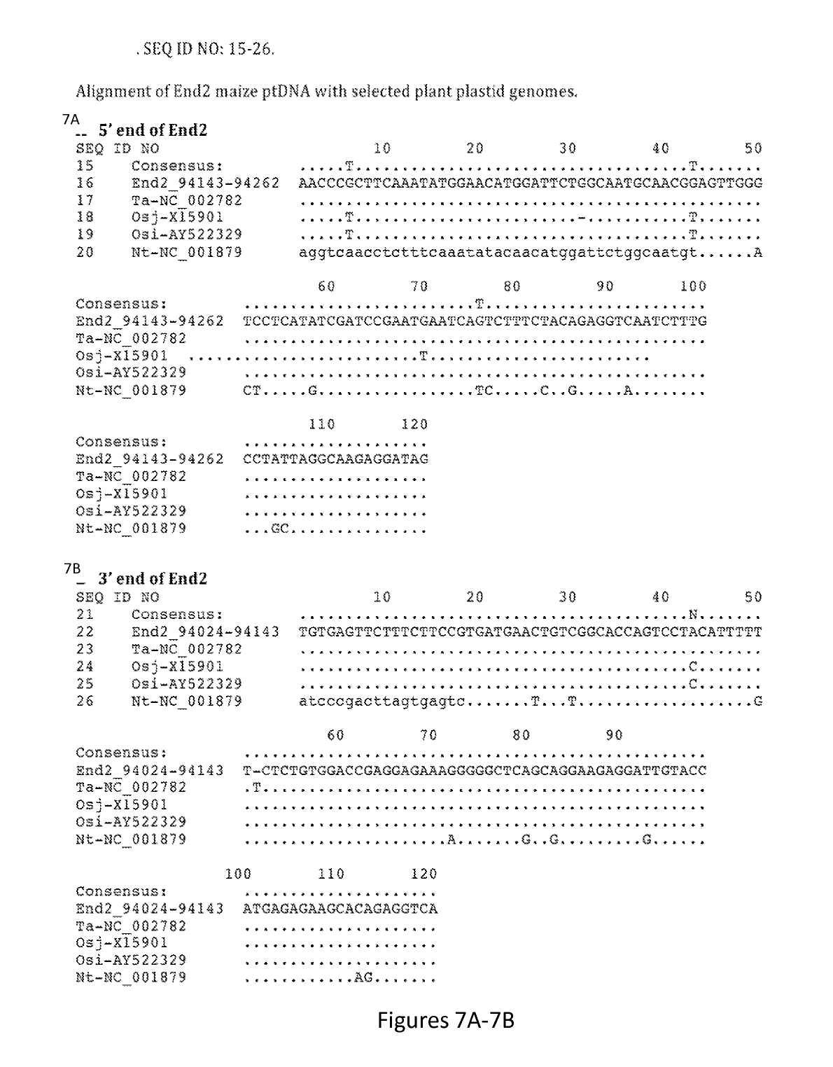

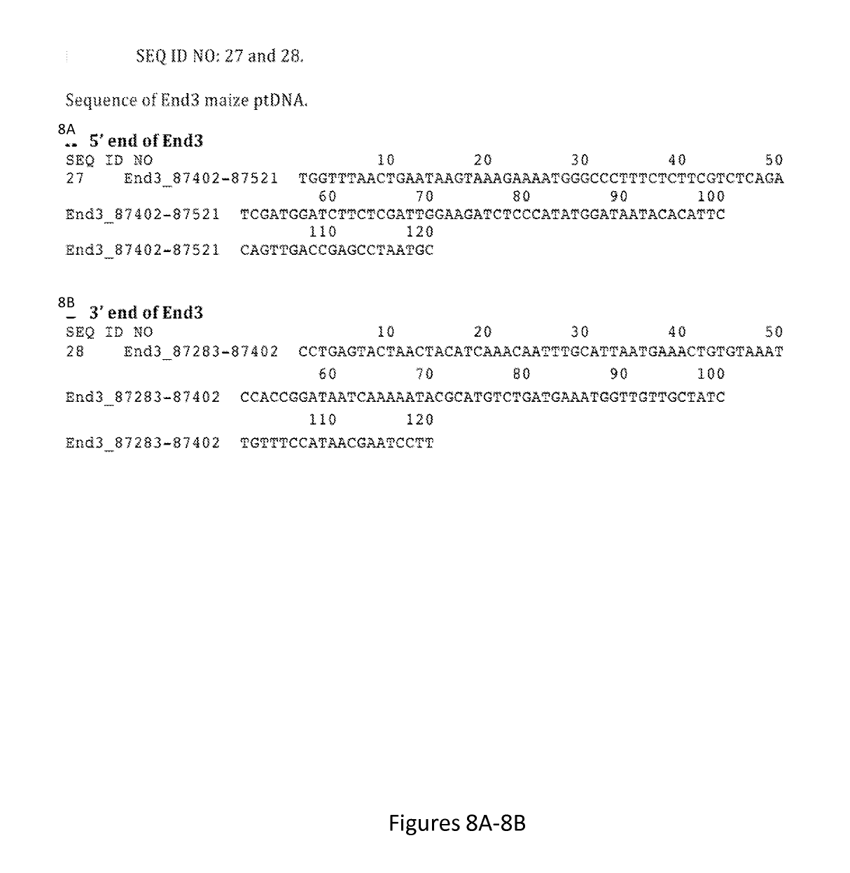

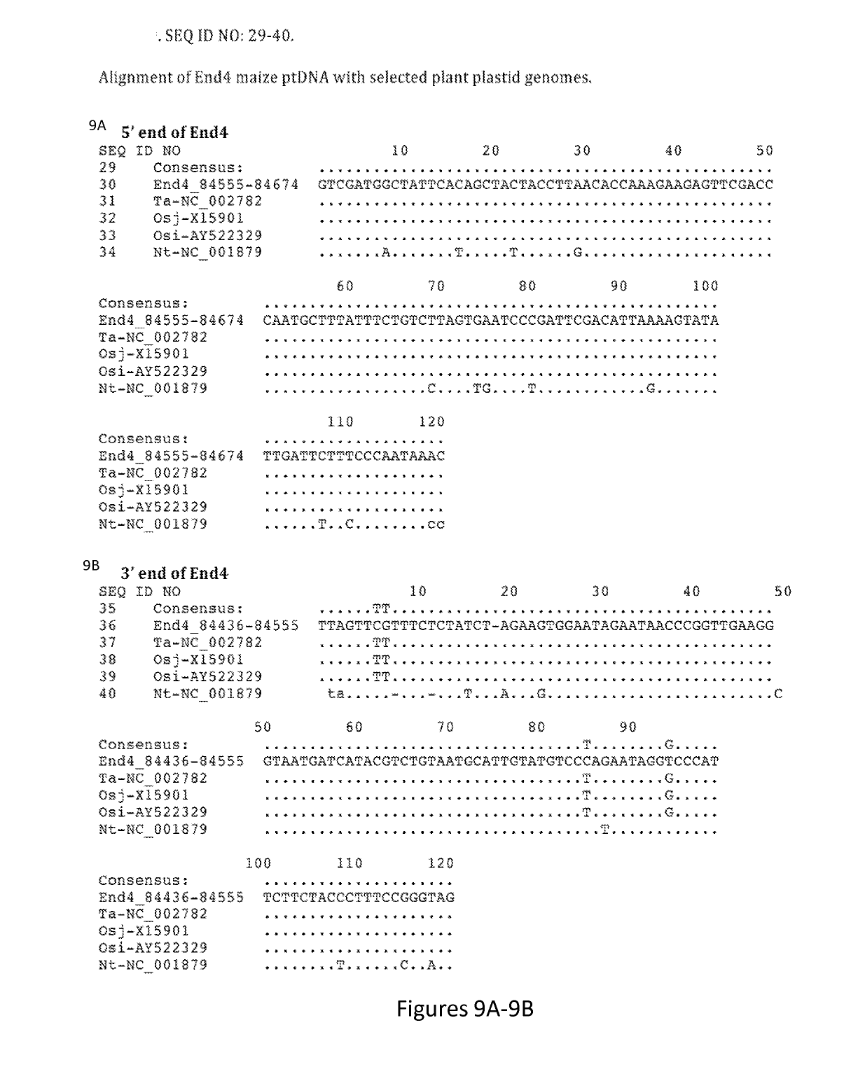

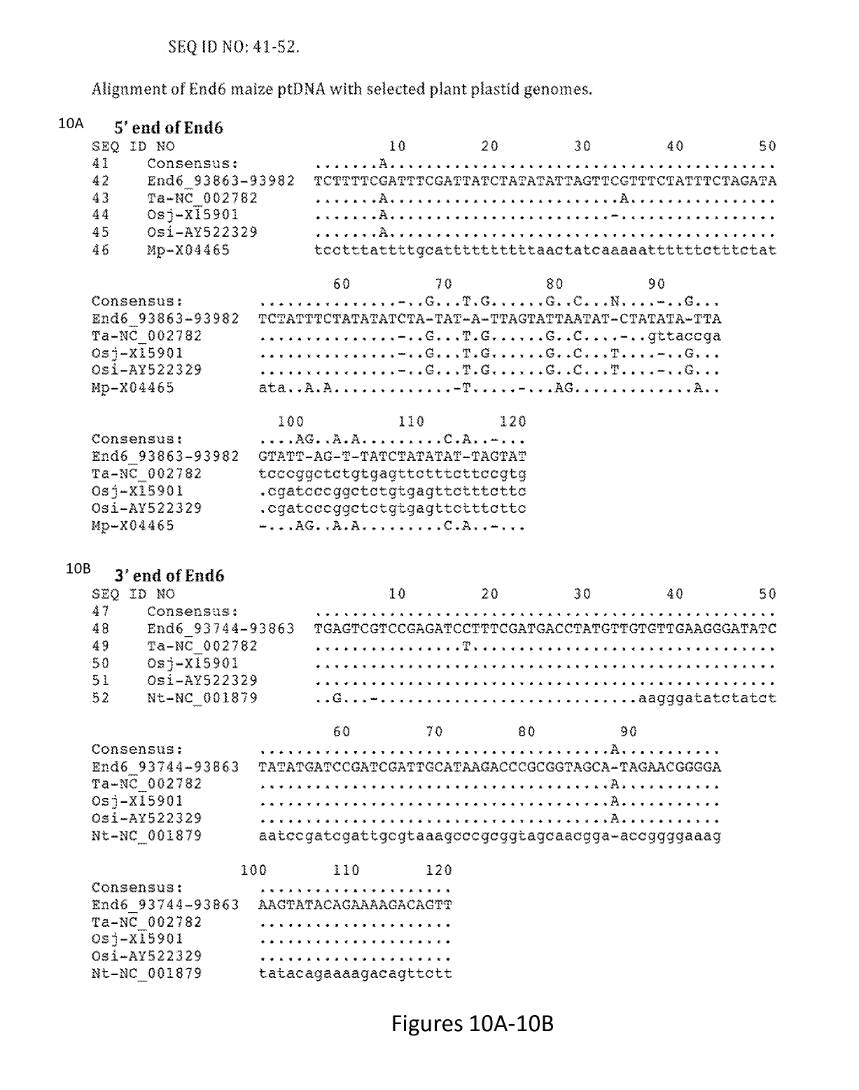

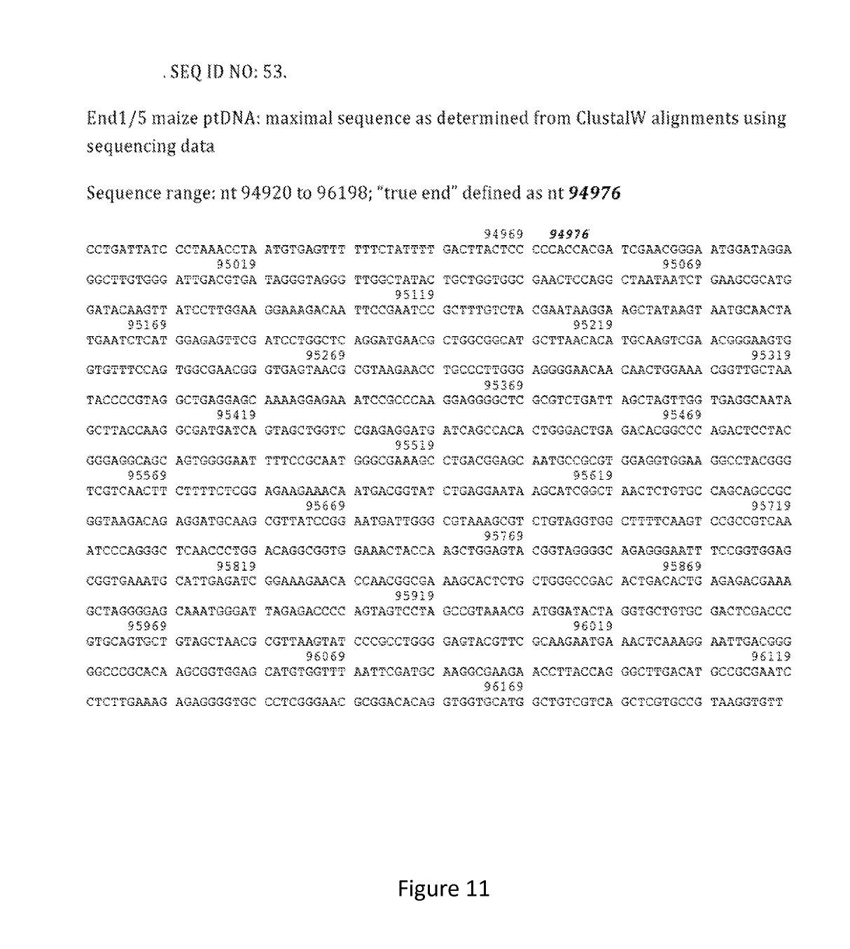

Portions of exemplary plastid end sequences include SEQ ID NOS:2-6 (see, FIG. 6). Portions of additional exemplary naturally occurring plastid end sequences include SEQ ID NOS:16-20 and 22-26 (FIG. 7), 27 and 28 (FIG. 8), 30-34 and 36-40 (FIG. 9), and 42-46 and 48-52 (FIG. 10). Other exemplary plastid end sequences may include any portion of the entire plastid terminal sequences given in FIGS. 11-15. A “plastid terminal sequence” can be broadly defined as one identified by the procedures described in Example 3. A “true end” is narrowly defined as the first nucleotide where the greatest degree of sequence overlap was found among all individual sequencing outputs for a specific end (Ends 1/5, 2-4, and 6 as given in Table 6).

In certain embodiments, a plastid terminal sequence comprises a portion of SEQ ID NO:1, 2, 3, 4, 5, 6 or 7, which portion is at least 30 nucleotides in length. Additional exemplary plastid terminal sequences comprise a portion of SEQ ID NO:8-52, which portion is at least 30 nucleotides in length.

When comparing polynucleotide sequences, two sequences are said to be “identical” if the sequence of nucleotides in each of the two sequences is the same when the sequences are aligned for maximum correspondence. The percentage identity between two nucleotide sequences as described herein (e.g., a plastid terminal sequence and a naturally occurring plastid end sequence) is determined according to MacVector using Align to Reference and ClustalW alignments (MacVector, Inc, http://www.macvector.com/; Larkin et al., Bioinformatics 23:2947-2948, 2007; http://www.clustal.org/).

In certain embodiments, the linear DNA vector may comprise a plastid terminal sequence as well as at least one other plastid targeting sequence homologous to a portion of plastid chromosome of interest. In some embodiments, the linear DNA vector may comprise a plastid terminal sequence and two or more additional plastid targeting sequences homologous to portions of plastid chromosome of interest.

In addition to a plastid DNA targeting sequence, a linear DNA vector may comprise a transgene of interest. Transgenes of interest include those encoding industrially valuable enzymes, biomaterials, therapeutic or prophylactic polypeptides, antibodies, antibiotics, vaccine antigens, genes that provide or enhance herbicide resistance, insect resistance, fungal resistance, bacterial resistance, drought tolerance, salt tolerance, cold and frost tolerance, and genes that improve nitrogen fixation, mineral nutrition, plant yield, starch accumulation, fatty acid accumulation, protein accumulation, phytoremediative ability, improved vigor, color or aesthetic appeal, health and nutritional characteristics, storae characteristics, heavy metal tolerance, water-stress tolerance, sweetness, taste, texture, decreased phosphate content, germination, micronutrient uptake, starch composition, and photosynthesis (Bock, Curr Opin Biotechnol 18:100-6, 2007; Bock and Warzecha, Trends Biotechnol 28:246-52, 2010; Daniell et al., Vaccine 23:1779-83, 2005; Daniell et al., Trends Plant Sci 14:669-79, 2009; Grevich and Daniell, Crit. Rev. Plant Sci. 24:83-108, 2005; Maliga, Annu Rev Plant Biol 55:289-313, 2004; Verma and Daniell, Plant Physiol 145:1129-43, 2007). Exemplary transgenes include cry, nif, tetC, and xynA. In certain embodiments, a transgene of interest is a selection marker gene.

In certain embodiments, a transgene is a gene that is originated from a species different from the plant species to which the transgene is introduced (e.g., another plant species or a different organism). In other embodiments, a transgene is a gene that is originated from the same plant species to which the transgene is introduced, but has been substantially modified from its native form in composition and/or genomic locus. In some embodiments, a transgene is a gene that is originated from the same plant species to which the transgene is introduced, but is not naturally present in plastids. In certain embodiments, a transgene is generated externally, such as a DNA sequence containing an antisense version of a gene. All of these types of transgenes may be referred to as a “heterologous” gene.

The transgene may be in an expression cassette in the linear DNA vector at a DNA insertion site. The expression cassette may comprise a promoter sequence operably linked with a downstream sequence (e.g., a transgene), a transcription termination sequence, and/or a gene encoding a selection marker (or a gene encoding a second selection marker if the transgene is a selection marker itself). The term “operably linked” as used herein refers to a functional linkage between a promoter and/or a transcription termination sequence and a second sequence, wherein the promoter sequence initiates and mediates transcription of the nucleotide sequence corresponding to the second sequence and the transcription termination sequence terminates transcription of the nucleic acid encoding the second sequence. Generally, operably linked means that the nucleic acid sequences being linked are contiguous and, where necessary to join two protein coding regions, contiguous and in the same reading frame. The promoter sequence (and/or the transcription termination sequence) may be native, analogous, foreign or heterologous to the host organism and/or to the transgene. Additionally, the promoter may be the natural sequence or alternatively a synthetic sequence.

The promoter may be any promoter that is active in the plastids of the plant species to be transformed. Exemplary promoters include chloroplast specific ribosomal RNA operon promoter rrn (16S rRNA), PEP promoter, psbA promoter (Staub et al., EMBO J 12:601-6, 1993), rbcL promoter, trnV promoter, rps16 promoter, and the promoter of the gene encoding the D1 thylakoid membrane protein. Certain exemplary promoters are provided in the Examples.

The transcription termination sequence may be any transcription termination sequence that is active in the plant species to be transformed. Exemplary transcription termination sequences include the psbA termination sequence and the termination sequences of rrn, rbcL, trnV, and rps16. Certain exemplary terminators are provided in the Examples.

As used herein “active in the plastids of the plant species to be transformed” indicates that the functional unit (e.g. a promoter or termination sequence) referred to can demonstrate a detectable level of activity in plastids of that plant species (e.g. when the functional unit is operably linked to a transgene and other required functional units in a plastid of the plant species, a detectable level of an expression product (e.g. RNA or polypeptide expression product) and/or, in the case of a terminator, a detectable level of properly-terminated copies of the transcript of that transgene can be detected under at least one set of conditions). A functional unit can be active in the plastids of the plant species if it is active in one or more type of plastid, e.g. one type of plastid, two types of plastid, or more types of plastids, up to and including all types of plastids. A functional unit can be active in the plastids of the plant species if it is active in one or more type of plastid in at least one stage of development, e.g. in mature plants, in seedlings. A functional unit can be active in the plastids of the plant species if it is active in one or more type of plastid in at least one tissue.

Additional untranslated region (UTR) sequences, fused to coding sequences of a transgene and/or a gene encoding a selection marker, can also be included in a linear DNA vector. Exemplary UTR sequences are provided in the Examples. Additional elements for expression of a protein, such as transcriptional and translational enhancer, ribosome binding sites and the like may also be included in the expression cassette.

The selection markers can include, but are not limited to, genes encoding polypeptides that confer resistance to spectinomycin, streptomycin, kanamycin, hygromycin, chloramphenicol, glyphosate, bialaphos, gentamycin or mannose. In some embodiments, a gene encoding a selection marker can be aadA that confers resistance to spectinomycin and streptomycin.

Alternatively, a visual marker can be used, such as a fluorescent protein. Exemplary visual markers include green fluorescence protein (GFP), β-glucuronidase (GUS), and luciferase (LUX). The selection may be made visually by illuminating the putative transformants with an appropriate source of light and selecting the transformants that show fluorescence.

In some embodiments, the selection marker can be a removable and/or excisable selection marker (e.g. “clean gene” technology), e.g. it can be flanked by LoxP sites such that it is excised from a host nucleic acid sequence in the presence of the Cre recombinase. Other recombinase systems are known in the art, e.g. Flp-Frt. Methods of making and using removable and/or excisable selection markers are known in the art, see, e.g. Day and Goldschmidt-Clermont. Plant Biotechnology 2011 9:540-533; which is incorporated by reference herein in its entirety.

The in vivo form of linear ptDNA molecules may include a telomeric (terminal) structure. This telomeric structure may be comprised of a blunt end, 5′ overhang, or 3′ overhang. Furthermore, the termini may have a structural modification, including a single-stranded loop that may or may not form a covalent bond joining the two DNA strands or a molecule, such as a protein, a peptide, or an amino acid covalently attached to the 5′ end or 3′ end. Modification of the linear DNA vector such that it has a telomeric structure similar to in vivo ptDNA may improve plastid transformation efficiency.

The linear DNA vector may have a single-stranded overhang at the 5′ end, the 3′ end or both the 5′ and 3′ ends. Alternatively, the linear DNA vector does not contain any single-stranded overhang at the 5′ end or the 3′ end.

In some embodiments, the linear DNA vector may have a single-stranded loop that may or may not covalently join the two DNA strands of the linear DNA vector.

In some other embodiments, the linear DNA may have a molecule that is not a nucleotide (e.g., a protein, a peptide or an amino acid) covalently joined to either the 5′ end or the 3′ end.

Plastids to which a linear DNA vector that comprises a transgene of interest may be introduced include any plastids that comprise substantially non-degraded plastid DNA. Exemplary plastids include etioplasts, proplastids, chromoplasts, leucoplasts, amyloplasts, elaioplasts, and chloroplasts containing substantially non-degraded plastid DNA. Types of plastids and methods of identifying them are known in the art; see, e.g. Wise. Advances in Photosynthesis and Respiration 2006 23:3-26; which is incorporated by reference herein in its entirety. In some embodiments, the plastid is a non-green plastid, e.g. it is not a chloroplast.

The complete suite of segregating genomic DNA sequence present in a plastid is referred to as a “unit genome”. Multiple copies of the unit genome may exist in any given plastid. In some embodiments, copies of any given portion of the unit genome, up to and including the entire unit genome, can exist within the same nucleic acid molecule. Accordingly, the length of a given undegraded nucleic acid molecule in a plastid can be x bp, 2x bp, 3x bp, etc. A nucleic acid molecule of x by of length can be referred to as “unit-genome sized” while a “multigenome sized” molecule could be of 2x bp, 3x bp, 4x bp, 5x by etc., in length. Development of the plastid and/or exposure to light can induce degradation of the plastid DNA, such that the given nucleic acid molecule is yx by in length where y is not a whole integer. In some embodiments, a plastid comprises substantially non-degraded plastid DNA if more than 25% of the plastid DNA is unit-genome and multigenomic sized. In certain embodiments, more than 30% to 90%, such as more than 30%, 35%, 40%, 45%, 50%, 55%, 60%, 70%, 80% or 90% of the plastid DNA is unit-genome and multigenomic sized. In some embodiments, a plastid comprising substantially non-degraded plastid DNA can be a plastid comprised by a non-green tissue. As used herein, a “non-green” cell or tissue is a cell or tissue comprising less than 20% of the chloroplasts and/or chlorophyll found in a green, photosynthetic cell or tissue (e.g. a green leaf), e.g. 20% or less, 10% or less, 5% or less, 2% or less, or 1% or less.

Any tissue that comprises substantially non-degraded plastid DNA may be used for plastid transformation. Such tissues include non-green tissues such as stalks and leaves of dark-grown seedlings, immature and mature embryos, seeds, dark-grown embryogenic callus, protoplasts, roots, tubers, and portions thereof. A mature embryo is the part of a fully-developed seed composed of one or two cotyledons, a radicle, and a hypocotyl. An immature embryo is part of a developing seed, typically harvested 11-14 days post pollination.

Using non-green tissues, especially those from cereals, for plastid transformation is advantageous over using green tissues. Without wishing to be bound by theory, the present inventors hypothesize that previous failures in attempts to transform plastids in cereals are at least partially due to the fact that DNA in certain green plastids (e.g., maize green plastids) is highly degraded. The present inventors further hypothesize that the previous failures may also result from the fact that cells containing green plastids have already differentiated from their precursors in meristematic cells and cannot pass plastid DNA onto their progeny cells. Thus, in certain embodiments, the tissues in which plastid transformation is performed are non-green tissues in an undifferentiated state. In some embodiments, most of plastids in the tissues are proplastids, e.g. at least 50% of the plastids are proplastids, at least 60% of the plastids are proplastids, at least 70% of the plastids are proplastids, at least 80% of the plastids are proplastids, or at least 90% of the plastids are proplastids.

Plants suitable for plastid transformation using the methods disclosed herein include dicotyledons and monocotyledons. In some embodiments, the plants are crop plants (e.g., cultivated plants including crops grown primarily for human consumption such as cereal crops), vegetables, fruits, seed crops, and oil plants. Cereal crops are grasses (members of the monocot family Poaceae), cultivated for the edible components of their grain composed of the endosperm, germ and bran. Cereal crops suitable for plastid transformation include maize, wheat, rice, barley, sorghum, millet, oats, triticale, rye, buckwheat, fonio, and (grain-like) quinoa. Additional exemplary plants that are suitable for plastid transformation include tobacco, lettuce, cotton, soybean, tomato, and Arabidopsis. It is contemplated that additional plants and/or crops are suitable for plastid transformation in accordance with the methods and compositions described herein. These additional plants and/or crops can include, but are not limited to: angiosperm and gymnosperm plants such as acacia, alfalfa, apple, apricot, artichoke, ash tree, asparagus, avocado, banana, beans, beet, birch, beech, blackberry, blueberry, broccoli, brussels sprouts, cabbage, canola, cantaloupe, carrot, cassava, cauliflower, celery, chestnut, cherry, chinese cabbage, citrus, clementine, clover, coffee, corn, cotton, cowpea, cucumber, eggplant, elm, endive, eucalyptus, fennel, figs, fir, geranium, grape, grapefruit, groundnuts, ground cherry, hickory, kale, kiwifruit, kohlrabi, larch, leek, lemon, lime, locust, maidenhair, mango, maple, melon, mushroom, mustard, nectarine, nuts, oak, okra, onion, orange, an ornamental plant or flower or tree, papaya, palm, parsley, parsnip, pea, peach, peanut, pear, peat, pepper, persimmon, pigeon pea, pineapple, plantain, plum, pomegranate, poplar, potato, pumpkin, quince, radicchio, radish, rapeseed, raspberry, sallow, spinach, squash, strawberry, sugarbeet, sugarcane, sunflower, sweet potato, sweet corn, tangerine, tea, trees, turf grasses, turnips, a vine, walnut, watercress, watermelon, yams, yew, zucchini, cedar, cypress, pine, sequoia, spruce, cultured cells of liverwort and related bryophytes, and cultured algae. The plant can be from a genus selected from the group consisting of Allium, Antirrhinum, Asparagus, Atropa, Avena, Beta, Brassica, Bromus, Browallia, Capsicum, Chenopodium Cichorium, Citrus, Cucumis, Cucurhita, Datura, Daucus, Dendranthema, Fragaria, Geranium, Glycine, Gossypium, Helianthus, Hemerocallis, Hordeum, Hyoscyamus, Juglans, Kalanchoe, Lactuca, Linum, Lolium, Lotus, Talus, Manihot, Medicago, Nicotiana, Onobrychis, Oryza, Panicum, Pelargonium, Pennisetum, Petunia, Pharbitis, Phaseolus, Pisum, Populus, Ranunculus, Raphanus, Rosa, Salpiglossis, Secale, Senecio, Sinapus, Solanum, Trifolium, Trigonella, Triticum, Vigna, Zea, Picea, Pinus, and Pseudotsuga.

Introducing a linear DNA vector into a plastid of a plant tissue may be performed by any techniques known in the art. Exemplary techniques include electroportation, particle gun transformation, polyethylene glycol transformation and whiskers technology (see, U.S. Patent Application Publication No. 2010/0218277; which is incorporated by reference herein in its entirety). In a preferred embodiment, a linear DNA vector is introduced into a plastid of a plant tissue via biolistic bombardment of the plant tissue with microparticles coated with the linear DNA vector.

After plastid transformation, cells having a transgene introduced into their plastids (e.g., having a transgene integrated into their plastid DNA) may be detected by various techniques known in the art, such as hybridization, electrophoresis, sequencing, and/or PCR. In certain embodiments, cells having a transgene introduced into their plastids may be selected via a selection marker whose gene is also introduced with the transgene. For example, if aadA is introduced with a transgene, streptomycin or spectinomycin may be used to screen for cells containing aadA and the transgene. Certain exemplary methods for detecting cells containing transgenes incorporated in their plastid DNA are provided in the Examples.

In certain embodiments, after plastid transformation treatment (e.g., biolistic bombardment coated with a linear DNA vector), the treated plant tissue is further cultured for 3 to 21 days without light and/or without being subject to any selection agent (e.g., streptomycin or spectinomycin). Delays in exposing the tissue to selection pressure prevent killing of many or most potentially transformable cells by the selection agent and increase transformation rates. Using non-green tissues and delaying their exposure to light (which is required for selection by certain selective agents, such as streptomycin or spectinomycin) also prevents certain plastid DNA (e.g., maize plastid DNA) from being rapidly degraded when exposed to light.

In a related aspect, the present disclosure provides a transplastomic plant or plant tissue obtained by the methods provided herein. A “transplastomic” plant or plant tissue refers to a plant or plant tissue having a heterologous gene (e.g. a transgene) introduced into at least a portion of its plastids. In preferred embodiments, the transplastomic plant or plant tissue is homoplasmic. A “homoplasmic” transplastomic plant or plant tissue contains only plastids carrying incorporated transgenes as part of their DNA and does not contain plastids whose DNA does not carry the transgene (i.e. plastids whose DNA comprises only wild type plastid DNA).

Because plant cells contain a large copy number of plastid genomes, an effective selection marker and selection regime are important for selecting homoplasmic transformants. Selection strategies known in the art may be used. Examples of such strategies include the use of selection markers such as aadA for spectinomycin/streptomycin antibiotic resistance (Svab et al., Proc Natl Acad Sci USA 87:8526-30, 1990; Verma Maliga and Bock, Plant Physiol 155:1501-10, 2011), bar for herbicide resistance (White et al., Nucleic Acids Res 18:1062, 1989; Nakamura et al., Biosci Biotechnol Biochem 74:1315-9, 2010), and/or pmi for metabolic selection (Wright et al., Plant Cell Rep 20:429-36, 2001). At this stage, if required by a selection marker (e.g., those based on the aadA or bar gene), transformants may be transferred from dark to light.

Transformants that contain transgenes of interest in their plastids may be used to regenerate transplastomic plants. Any methods known in the art for regenerating transgenic or transplastomic plants may be used. Exemplary methods include regeneration of maize using somatic embrogenesis and seedling meristematic tissue (Santos et al., Plant Sci Letters 33:309-315, 1984; Zhong et al., Planta 187:483-489, 1992; Al-Abed et al., Planta 223:1355-60, 2006; Huang and Wei, Plant Cell Rep 22:793-800, 2004; Li et al., Theor Appl Genet 108:671687, 2004; Ahmadabadi et al., Transgenic Res 16:437448, 2007).

In another related aspect, the present disclosure also provides progenies of transplastomic plants or plant tissues. In certain embodiments, such progenies are homoplastomic.

In another aspect, the present disclosure provides a linear DNA vector that comprises (1) a plastid DNA targeting sequence that comprises a plastid terminal sequence, and (2) an expression cassette that comprises: (a) optionally a promoter active in the plastids of the plant to be transformed, (b) a DNA insertion site for receiving a transgene of interest, (c) optionally one or more selection markers, and (d) optionally a DNA sequence encoding a transcription termination region active in the plastids of the plant to be transformed. In certain embodiments, the linear DNA vector does not have a transgene of interest inserted at the DNA insertion site. Such a vector facilitates plastid transformation of different transgenes. In certain embodiments, a particular transgene of interest is already inserted at the DNA insertion site. Different components of the linear DNA vectors are described above in connection with methods of plastid transformation provided herein. The linear DNA vector may be obtained by linearizing a circular DNA vector that comprises various components as described above and is also capable of self replication in host cells (e.g., bacterial or yeast cells).

As used herein, the term “expression cassette” refers to a nucleic acid molecule capable of conferring the expression of a gene product when introduced into a plant host cell, e.g. host cell plastid.

As used herein, the term “plant” refers to any of various photosynthetic, eukaryotic, multicellular organisms of the kingdom Plantae characteristically producing embryos, containing chloroplasts, and having cellulose cell walls. A part of a plant, i.e., a “plant tissue” can be treated according to the methods described herein. According to the present invention “plant tissue” also encompasses plant cells. Plant cells include suspension cultures, callus, embryos, meristematic regions, callus tissue, leaves, roots, shoots, gametophytes, sporophytes, pollen, seeds and microspores. Plant tissues can be at various stages of maturity and can be grown in liquid or solid culture, or in soil or suitable media in pots, greenhouses or fields. A plant tissue also refers to any clone of such a plant, seed, progeny, propagule whether generated sexually or asexually, and descendents of any of these, such as cuttings or seed.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); and Davis et al., Basic Methods in Molecular Biology, Elsevier Science Publishing, Inc., New York, USA (1995); which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. The results in these examples show successful plastid transformation in liverwort, tobacco, maize, wheat, and rice using linear DNA vectors and non-green tissues.

Some embodiments of the technology described herein can be defined according to any of the following numbered paragraphs:
1. A method of plastid transformation, comprising: (a) introducing a linear DNA vector into a plastid of a plant tissue, wherein
   (i) the plastid comprises substantially non-degraded plastid DNA, and
   (ii) the linear DNA vector comprises
      (1) a plastid DNA targeting sequence, and
      (2) a transgene of interest.
2. The method of paragraph 1, wherein the plant is a monocot or a dicot.
3. The method of any of paragraphs 1-2, wherein the plant is a liverwort or related species.
4. The method of any of paragraphs 1-3, wherein the transgene of interest is selected from the group consisting of genes encoding therapeutic or prophylactic polypeptides, genes that provide or enhance herbicide resistance, insect resistance, fungal resistance, bacterial resistance, and stress tolerance, and genes that improve nitrogen fixation, mineral nutrition, plant yield, starch accumulation, fatty acid accumulation, protein accumulation, and photosynthesis.
5. The method of any one of paragraphs 1 to 4, wherein the linear DNA vector further comprises a gene encoding a selection marker.
6. The method of paragraph 5, wherein the selection marker is a gene providing resistance against spectinomycin, streptomycin, kanamycin, hygromycin, chloramphenicol, glyphosate or bialaphos.
7. The method of paragraph 5, wherein the selection marker is a gene providing metabolism of mannose.
8. The method of paragraph 5, wherein the selection marker is a gene encoding a fluorescent protein.
9. The method of any one of paragraphs 1 to 8, wherein the plastid DNA targeting sequence comprises a terminal sequence of a plastid chromosomal DNA molecule.
10. The method of paragraph 9, wherein the plastid terminal sequence is at least 90% identical to a portion of SEQ ID NO:1, 8, 15, 21, 29, 35, 41, or 47, which portion is at least 30 nucleotides in length.
11. The method of paragraph 9, wherein the plastid terminal sequence comprises:

at least 30 consecutive nucleotides of SEQ ID NO:2, 9, 16, 22, 27, 28, 30, 36, 42, 48, 53, 54, 55, 56, or 57 when the plant tissue is a maize tissue,
at least 30 consecutive nucleotides of SEQ ID NO:3, 10, 17, 23, 31, 37, 43, or 49 when the plant tissue is a wheat tissue,
at least 30 consecutive nucleotides of SEQ ID NO:4, 5, 11, 12, 18, 19, 24, 25, 32, 33, 38, 39, 44, 45, 50, or 51 when the plant tissue is a rice tissue,
at least 30 consecutive nucleotides of SEQ ID NO:6, 13, 20, 26, 34, 40, or 52 when the plant tissue is a tobacco tissue, or
at least 30 consecutive nucleotides of SEQ ID NO:7 or 46 when the plant tissue is a liverwort tissue.
12. The method of any of paragraphs 1-11, wherein the linear DNA vector comprises a single-stranded overhang at either the 5′ end or the 3′ end, a single-stranded loop that may or may not covalently join the two DNA strands of the linear DNA vector, or a molecule that is not a nucleotide covalently joined to either the 5′ end or the 3′ end.
13. The method of any one of paragraphs 1 to 12, wherein the plant tissue is a non-green tissue.
14. The method of paragraph 13, wherein the non-green tissue is a portion of a mature embryo, a portion of a dark-grown seedling, a seed, or a portion of a seed.
15. The method of any one of paragraphs 1 to 14, wherein the plastid is a proplastid, etioplast, or other non-green plastid.
16. The method of any one of paragraphs 1 to 15, wherein step (a) is performed via biolistic bombardment of the plant tissue with microparticles coated with the linear DNA vector.
17. The method of any one of paragraphs 1 to 16, further comprising (b) culturing the plant tissue from step (a) without light.
18. The method of any one of paragraphs 1 to 17, further comprising (c) regenerating a transplastomic plant from the plant tissue from step (a) or step (b).
19. The method of paragraph 18, wherein the transplastomic plant is homoplasmic.
20. A transplastomic plant or a plant part obtained by the method of any one of paragraphs 1 to 19.
21. A progeny of a plant or plant part of paragraph 20.
22. A linear DNA vector for plastid transformation in a plant, comprising:
   (1) a plastid DNA targeting sequence that comprises a plastid terminal sequence, and
   (2) an expression cassette that comprises:
      (a) optionally a promoter active in the plastids of the plant to be transformed,
      (b) a DNA insertion site for receiving a transgene of interest,
      (c) optionally one or more selection markers, and
      (d) optionally a DNA sequence encoding a transcription termination region active in the plastids of the plant to be transformed.
23. The linear DNA vector of paragraph 22, further comprising a transgene of interest inserted at the DNA insertion site.

### EXAMPLES

### Example 1

### Plastid Transformation in Liverwort

A circular plastid transformation vector, pCS31, and its linearized form were used in transforming liverwort plastids. pCS31 comprises a standard E. coli plasmid (pBluescript II SK+), a right and left border sequence (RBS and LBS, respectively) homologous to a region of the plastid genome, and the transgene (aadA) (FIG. 1A) (Chiyoda et al., Transgenic Res 16:41-9, 2007). The plastid DNA (ptDNA) region corresponds to nucleotides (nt) 83,881-85,894 of IRb and 116,226-118,239 of IRa (GenBank Accession No. X04465) and contains the trnI and trnA genes. Restriction digestion produces a linearized form (LpCS31) with an end at either the RBS or LBS (FIG. 1B) or just the transgene cassette (TCpCS31), which includes one or both border sequences and the transgene (FIG. 1C). For most of the liverwort cell experiments, a TCpCS31 vector containing only RBS plus the transgene was used. The end of the RBS in both LpCS31 and TCpCS31 is ˜3000 bp from the liverwort ptDNA sequence predicted to be analogous to Zm End1/5 (Tables 6 and 7).

Liverwort cell-suspension cultures were grown in 1M51C medium and subcultured every two weeks (see, Oldenburg and Bendich, J Mol Biol 310:549-62, 2001; Oldenburg and Bendich, J Mol Biol 276:745-58, 1998). For plastid transformation, cells were sterile-filtered onto 42.5-mm Whatman disks and placed onto 1M51C agar plates. Cells were then bombarded with 0.6 μm gold microcarriers coated with either circular or linearized vector (0.5-1 μg DNA). After a 48-hr recovery period, the cells/disks were placed on spectinomycin selection plates (1M51C+500 μg/mL spectinomycin) for 3-4 weeks under fluorescent lights. Some colonies were transferred from the primary (1°) selection plates to fresh plates containing spectinomycin for secondary (2°) selection screening. For 2° selection, either a single colony or 2-5 colonies were chosen, resuspended in liquid 1M51C medium, and spread onto selection plates.

Transformation efficiency with liverwort was assessed from the number of green colonies on spectinomycin selection medium (Table 1), and the presence of the transgene was assessed by PCR and dot blot hybridization to the aadA gene (Table 2). A higher transformation efficiency (usually 3-10-fold, but 200-fold in one case; Table 1) was found with the linearized vector than with the circular vector.

The conventional method of transgene integration using a circular vector is by homologous recombination (HR) between the vector and homologous regions (LBS and RBS) within the ptDNA (FIG. 1D). In contrast, a linear vector may integrate by HR, EJ (end joining; FIG. 1E), or SI (strand invasion; FIG. 1F). In the case of the linear transgene cassette (TCpCS31), integration would be by EJ or SI, but not HR since it only contains one region (RBS) homologous to the ptDNA (FIG. 1). PCR amplification products obtained with primers P3/P6 would indicate positive transformants via strand-invasion, but not if integration is via EJ. The aadA primers aL/aR, on the other hand, would report all positive transformants regardless of mechanism. Thus, the PCR data with P3/P6 primers (lower number with TCpCS31 compared to aadA primers; Table 2) implies integration by EJ, not SI. For primary TCpCS31, 21% of integrations are by SI and 79% are by EJ relative to the total number of aadA positive transformants (4/19 and 15/19, respectively). Table 3 presents the mechanisms used for transgene integration using the linear DNA vector.

### Example 2

### Plastid Transformation in Tobacco

Plastid transformation in tobacco was conducted with young, expanding leaves and older, fully-expanded leaves using both spectinomycin selection (aadA gene) and the visual marker, gfp. Using the aadA vector, a positive transformant was scored as one with some callus-like growth on a leaf segment. Such tissue was either pale-yellow or green with trichomes often protruding from the tissue. The selection medium contained hormones that should permit growth of shoots (in addition to spectinomycin selection); however, no shoots were obtained. Plastid transformation was 4- to 6-fold better with young than older leaves (Table 4). Positive transformants with GFP expression in plastids were also found with the linear gfp vector (images not shown).

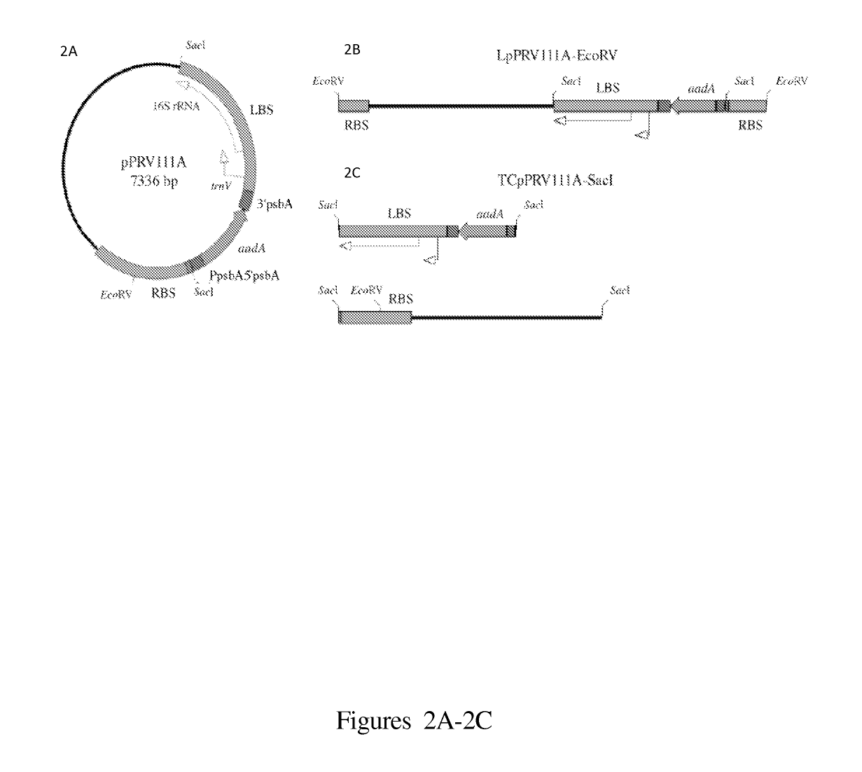

Nicotiana tabacum Petite Havana was grown aseptically in RM agar in a controlled temperature room under continuous light. Leaves of varying age were harvested and measured before placing abaxial side up on RMOP plates. Leaves were then bombarded with 0.7 μm tungsten microcarriers coated with either circular or linearized pPRV111A (FIG. 2) or pPRV131B vectors (Lutz et al., Plant Physiol 145:1201-10, 2007). Leaves were left over night and the next day were cut into 5×5 mm sections and placed onto RMOP selection plates containing 500 mg/L spectinomycin, abaxial side on medium. The RMOP agar used had been changed from the original growth hormone concentrations to contain 0.5 mg/L NAA and 0.5 mg/L BAP. Three to four weeks later, leaf sections were scored for regeneration (Table 4). It is not unusual to have selection “escapes” using aadA/spectinomycin selection with tobacco (Svab and Maliga, Proc Natl Acad Sci USA 90:913-7, 1993) as shown by the presence of calli with the no-DNA leaf segments (Table 4). Leaves that had been bombarded using pPRV131B, which confers GFP expression, were also examined microscopically for GFP fluorescence (images not shown). PCR primers aadA R1/L1 were added to total tissue DNA (ttDNA), and amplified products were visualized as bands on agarose gels (Table 5).

Plastid transformation efficiency using the linear vector was slightly higher than with the circular vector. For TCpPRV111A, the EcoRVISacI digestion did not go to completion (agarose gel analysis, data not shown), yielding both LpPRV111A (FIG. 2B) and TCpPRV111A (FIG. 2C), but no circular vector. Slightly higher transformation was found with TCpPRV111A than with LpPRV111A, suggesting that a vector comprised of just one ptDNA-targeting region (RBS) plus the marker gene (aadA) may be better for integration into the plastid genome than a vector with both RBS and LBS. Furthermore, since liverwort plastid transformation indicates better integration by EJ than by HR, it is likely that the higher number of tobacco transformants was due to EJ for TCpPRV111A. The end of the RBS in TCpPRV111A is ˜1500 bp from the tobacco ptDNA sequence predicted to be analogous to Zm End1/5 (Tables 6 and 7).

### Example 3

### Plastid Transformation in Maize, Wheat and Rice

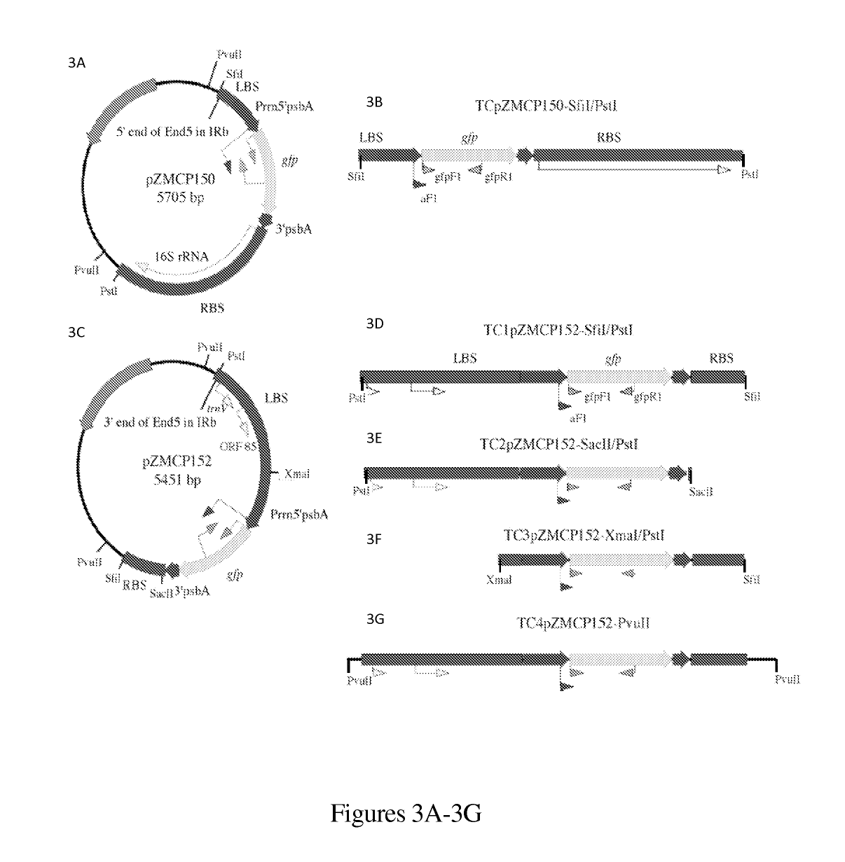

Plastid transformation vectors for maize that contain ptDNA end sequences and marker transgenes (gfp) were constructed (FIG. 3). The methods used and location of end sequences within the maize plastid genome are described below. Non-green maize tissues (mature embryos and stalk of dark-grown seedlings) as well as wheat and rice tissues (seeds either whole or split open to expose the embryo) were used for plastid transformation by particle bombardment with both circular and linearized vectors. Callus and developing shoot tissues were evaluated for gfp expression a few days after bombardment and tissue samples collected for total tissue DNA (ttDNA) preparation, PCR and blot-hybridization analysis.

1. Determination of end sequences for maize ptDNA. The size of the plastid genome is 140,387 bp in maize (Maier et al., J Mol Biol 251:614-28, 1995). Plastid chromosomal DNA molecules exist as collection of unit-genome-sized linear isomers (monomers and concatemers) and multigenomic, branched, complex molecules (Oldenburg and Bendich, J Mol Biol 335:953-70, 2004). The plastid genomes in maize and most, but not all, plants have four defined regions: a large single copy region (LSC), a small single copy region (SSC) and two inverted repeat regions (IRs; IRa and IRb). For sequenced plastid genomes, the first nucleotide (nt=1) is defined as the beginning of the LSC or its equivalent in genomes without IRs. The end sequences are regions adjacent to (either 5′ downstream or 3′ upstream) the Ends (nts), as described below.

The end sequences for maize ptDNA were determined using three methods: (1) one- and two-site restriction digestion followed by pulsed field gel electrophoresis (PFGE) and blot hybridization, (2) end-ligation to a cloning plasmid followed by PCR amplification of the ptDNA insert and sequencing, and (3) end-ligation to a cloning plasmid followed by subcloning into E. coli, plasmid selection/preparation and sequencing.

Using the first method, three discrete Ends for maize ptDNA were identified by restriction enzyme digestion and blot hybridization (Oldenburg and Bendich, J Mol Biol 335:953-70, 2004). Using the same methods, Ends have also been identified for ptDNA of Medicago truncatula (Shaver et al., Plant Physiol 146:1064-74, 2008) and tobacco (Scharff and Koop, Plant Mol Biol 62:611-21, 2006). This method showed that the Ends in the IRb of maize are located approximately at nts 78,000, 88,000, and 100,000, with standard deviation of ±5,000 bp (Ends are also in IRa).

For the other methods, a linearized plasmid, such as pBluescript, was ligated to the Ends of maize ptDNA as prepared by standard procedures (Oldenburg and Bendich, J Mol Biol 335:953-70, 2004). The in-gel plasmid-ligated-ptDNA was fractionated by PFGE, and the well-bound fraction (multigenomic, branched linear form) and linear monomer fraction were excised from the gel. The next step was to digest the plasmid-ligated-ptDNA with a restriction enzyme (such as BamHI) to give compatible ends within the plasmid polylinker and the ptDNA, followed by ligation of the compatible ends. Two methods were then employed to sequence the ends adjacent to the plasmid using universal primers (M13 forward and reverse). The first was PCR using M13 primers to amplify the ptDNA insert that includes the End regions. Agarose gel electrophoresis was used to fractionate the PCR products, followed by excision of DNA bands and sequencing. The second method was E. coli transformation with the plasmid-ptDNA, selection of colonies, plasmid minipreps and sequencing of the plasmid with the ptDNA insert that includes the End regions. These procedures were performed using ptDNA from several different maize ptDNA preparations and on both branched linear (well-bound) and linear monomer molecules. The location of five End sequences was determined using these two methods (Table 6 and FIGS. 11-15). The terminus of End3 is at nt 87,402 and likely corresponds to an end (nt 88,000) previously identified by restriction digestion and blot hybridization. End1/5 was used to construct vectors (see below; FIG. 3) for maize, wheat, and rice plastid transformation.

For liverwort and tobacco, the plastid genomes and transformation vectors were evaluated for sequence similarity to the maize ptDNA ends (Tables 6 and 7). Sequence homology was found for all maize ends, with near-exact end sequences (>90% identity) in tobacco for three of the five maize ends and two of the five for liverwort (although one of these is in the LSC not the IRs). As shown in more detail, substantial sequence homology to maize ptDNA End1/5 was found among ptDNAs of wheat, rice and tobacco, with a shorter region (about 40 bp) of homology in liverwort ptDNA (FIG. 6). Interestingly, the ends of the linearized vectors used for liverwort and tobacco plastid transformation are near putative end sequences, as determined by comparison to the maize ends (Tables 6 and 7).

2. Vector constructs for plastid transformation of maize, wheat, and rice. Two vectors were constructed and tested for plastid transformation in maize, wheat, and rice (FIG. 3, A-D). The vectors are comprised of LBS and RBS regions, a marker transgene, and expression control regions that are inserted into the cloning plasmid pBluescript II KS+(Table 8). Plasmids were constructed with each of these three regions individually and with unique restriction sites to allow modular assembly and testing of multiple plastid transformation vectors. In addition, codons were optimized for expression in maize plastids. Plasmid pZMCP120 contains the marker transgene gfp (pzmcpGFP) flanked by maize ptDNA expression regulatory sequences for the 16S rRNA promoter+5′-UTR of psbA (nts 94976:95095 in IRb and 1151:1311 in LSC, respectively) and the 3′-UTR of psbA (nts 1:88 in LSC) (pZmPrrnpsbA53). Plasmid pZMCP112 contains the LBS1/RBS1 sequence corresponding to nts 94976:96740 of IRb and is 5′ downstream of End5 (pZMCP112). For plastid transformation vector pZMCP150, the regulatory/marker sequence was inserted at the SalI restriction site of pZMCP112. Plasmid pZMCP113 contains the LBS2/RBS2 sequence corresponding to nts 93476:94976 of IRb and is 3′ upstream of End5. For plastid transformation vector pZMCP152, the regulatory/marker sequence was inserted at the SalI restriction site of pZMCP113.

Four additional linear vectors were also generated by restriction digestion and tested for plastid transformation in maize, wheat, and rice (FIG. 3, E-G). TC2pZMCP152 includes entire LBS with End5 and minimal RBS (27 bp regions) (FIG. 3E). T3pZMCP152 includes entire RBS and minimal LBS (172 bp) without End5 (FIG. 3F). TC4pZMCP152 includes entire LBS and RBS regions, but the end sequences comprise 113 and 205 bp from the cloning plasmid (FIG. 3G), thus obscuring or “blocking” the “true end” of the ptDNA.

3. Methods for vector delivery, maize, wheat, and rice tissue culture, and plastid transformation. Seeds (Zea mays inbred B73, Triticum aestivum var. Chinese Spring wheat, or Oryza sativa japonica var. M104) were sterilized in 20% bleach (+one drop of Tween 20) for 30 min, then rinsed several times with sterile water. Seeds were soaked for a 2-5 days and maize embryos dissected. Either whole embryos or 3-4 mm pieces of maize were placed onto the center of N6 agar plates containing 1 mg/L 2,4-D and 0.5 mg/L BAP and placed in the dark. For wheat and rice, entire seeds either whole or split open to expose the embryo were placed on N6 plates. Particle bombardment was performed using either 0.3 or 0.4 μm gold microcarriers coated with circular or linearized vectors one day after whole embryos were plated or 3-10 days after plating for embryo pieces, by which time callus and/or shoots had developed.

For dark-grown seedlings of maize seeds were placed in Magenta boxes containing MS agar and grown in the dark for 1-3 weeks. The stalk (from basal meristematic node to ligule of first leaf, L1) was cut into 3-4 mm sections and placed onto N6 agar plates containing 1 mg/L 2,4-D and 0.5 mg/L BAP and kept in the dark for about 1 week. Biolistics was then performed using 0.3 μm gold microcarriers with circular or linear vectors. Plates with stalk tissue were kept in the dark and growth of callus, stem, and shoot tissue was discerned.

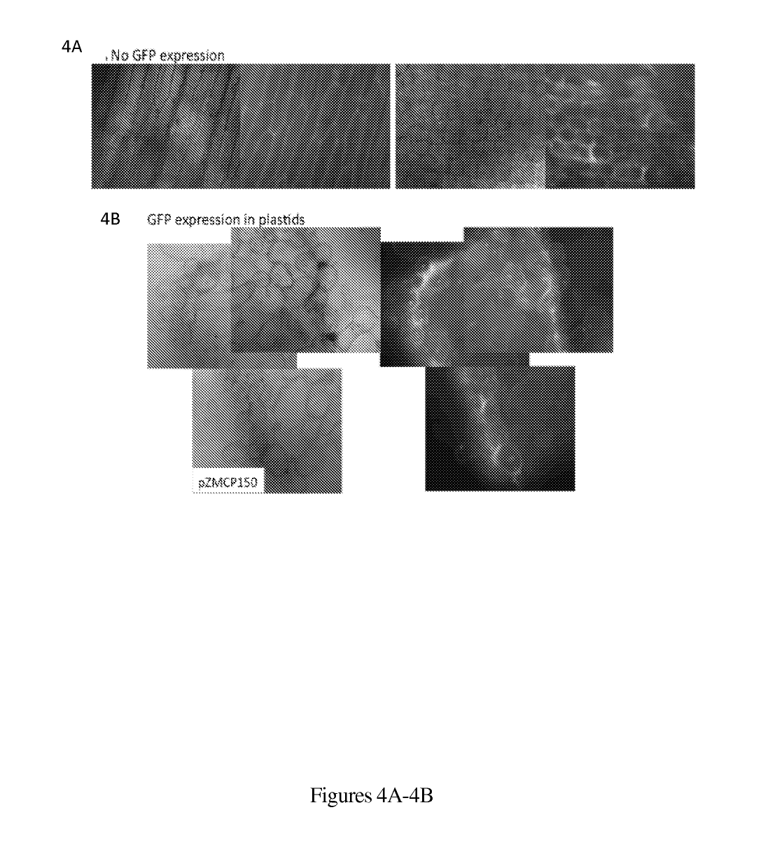

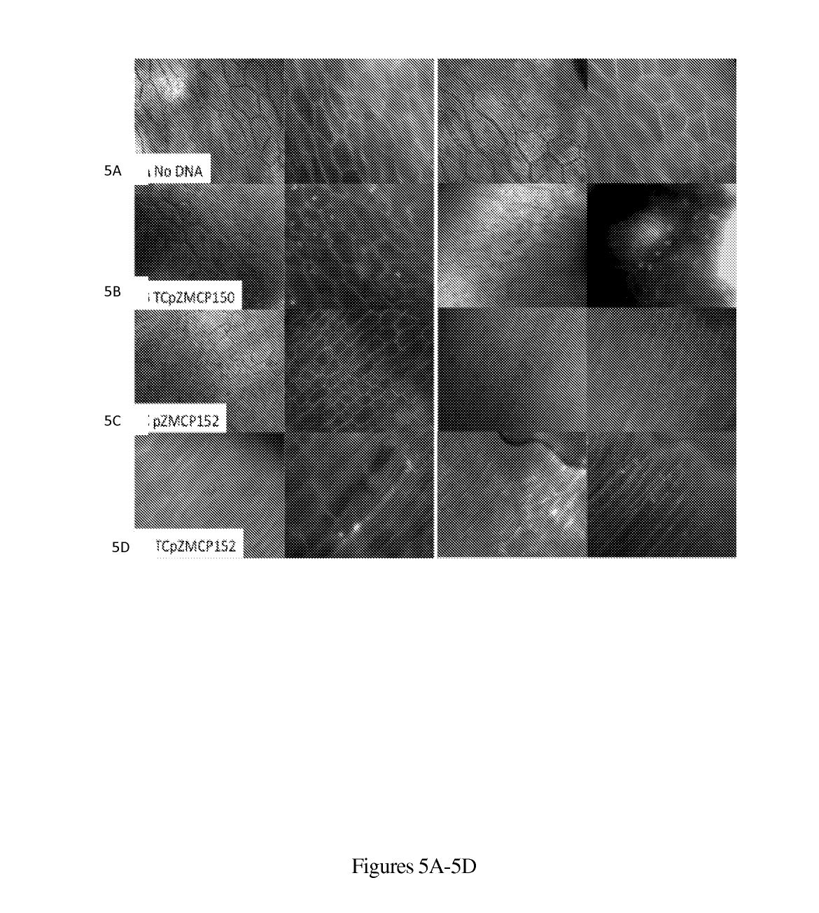

4. Plastid transformation results. Plastid transformation of maize tissue was assessed for GFP expression. Initial GFP expression was assessed by visual inspection using a hand-held laser (ex 405 nm) and a dissecting microscope equipped with a SYBR green filter (em 520 nm). Within 3 days after biolistics, sectors with putative GFP-plastid cells were discerned on some plates. In general, examination for GFP expression was performed 5-10 days after biolistics (Tables 9 and 10), and the position of positive tissues was marked on the bottom of the plates. Individual pieces of tissue (both positive and negative for GFP by laser) were selected for examination by fluorescence microscopy. Cells with GFP-plastids were observed in maize tissue transformed with both the circular and linear vectors (Table 10, FIGS. 4 and 5). To further assess transformation efficiency, several pieces of tissue from each transformation plate were processed for ttDNA and evaluated by PCR amplification with 03-specific primers and dot blot hybridization to a DIG-gfp probe (Table 9).

The efficiency of plastid transformation was assessed for a linear vector with a “true end” (FIG. 3D) and one where the end sequence is “blocked” by a “non-true end”, in this case a portion of the cloning plasmid (FIG. 3G). Protoplasts were prepared from the vector-transformed maize tissues and GFP expression assessed by fluorescence microscopy (Table 11). Approximately 2-fold greater transformation effeciency was found with the linear vector with a “true end” compared to both the circular and the “non-true end” linear vectors. Integration of the transgene may proceed by double-reciprocal homologous recombination for the “true end”, “non-true end” linear, and circular vectors. The “true end” linear vector, however, is also capable of integration via end joining or strand invasion.

Positive transformation of wheat and rice (Table 12) plastids was indicated by imaging of GFP-fluorescence and DIG-gfp dot blot hybridization of wheat ttDNA (Table 12) as described above for maize.

TABLE 1
Plastid transformation of liverwort cells

| | Total # | # Positive | Transformation |
|---|---|---|---|
| Vector | bombardments^{a} | transformants^{b} | efficiency^{c} |
| None | 10 | 2 (0-1) | 5 (0-5) |
| Circular - pCS31 | 13 | 38 (0-11) | 20 (0-50) |
| Linear - TCpCS31 | 21 | 512 (0-207) | 147 (0-997) |

^{a}Total number of bombardments: number of plates with liverwort cells used for particle bombardment.
^{b}Number of postive transformants following 1° selection: the average number of green colonies from all plates and range for individual plates is given.
^{c}Transformation efficiency: the average number of green colonies per gram cells (# transf/~0.2-0.3 g cells) from all plates and range for individual plates is given.

TABLE 2
PCR and blot hybridization assessment of liverwort plastid transgene integration

| | PCR primers^{a} | | | | | | Dot blot hyb^{b} | |
|---|---|---|---|---|---|---|---|---|
| | LWT-P5/P6 | | LWT-P3/P6 | | aadA-L1/R1 | | aadA | |
| Vector^{c} | #pos/total^{d} | % pos^{e} | #pos/total | % pos | #pos/total | % pos | #pos/total | % pos |
| none-1° | 0/4 | 0 | 0/4 | 0 | 2/4 | 50 | 0/2 | 0 |
| pCS31-1° | 1/7 | 14 | 2/7 | 28 | 4/7 | 57 | 0/7 | 0 |
| pCS31-2° | 6/9 | 67 | 4/9 | 44 | 8/9 | 89 | 7/9 | 78 |
| TCpCS31-1° | 9/28 | 32 | 4/28 | 14 | 19/28 | 68 | 17/28 | 61 |
| TCpCS31-2° | 9/29 | 31 | 14/29 | 48 | 26/29 | 90 | 22/29 | 76 |

^{a}Primer sets used for PCR amplification of total tissue DNA from putative transformants (see FIG. 1).
^{b}Dot blot hybridization of total tissue DNA from putative liverwort transformants with aadA gene probe.
^{c}Vectors used. Transformants from primary (1°) and secondary (2°) selection.
^{d}Number of transformants positive for PCR amplification or aadA hybridization/number of green colonies tested.
^{e}The percentage of positive transformants.

TABLE 3
PCR products and mechanism of transgene integration for liverwort
plastid transformation using TCpCS31 (aadA-RBS)

| | | Mechanism | | |
|---|---|---|---|---|
| | | of integration | | |
| Primers^{a} | Region amplified^{b} | HR | EJ | SI |
| LWT-P5/P6 | LBS-aadA-RBS | − | − | + |
| LWT-P3/P6 | RBS | − | − | + |
| aadA-L1/R1 | aadA | − | + | + |

^{a}PCR primer sets used to amplify transgene regions from total tissue DNA (ttDNA) prepared from liverwort tissue. Primers LWT-P5 and -P6 correspond to wild type ptDNA sequences that flank (are outside of) the integrated transgene.
^{b}Region of transgene within ptDNA amplified with PCR primer sets.
^{c}HR: homologous recombination; EJ: end joining; SI: strand invasion. Note: HR integration of transgene requires both LBS and RBS. Thus TCpCS31 does not integrate by this mechanism since it does not contain the LBS region. In contrast, transgene integration by HR does occur for circular pCS31 and linearized pCS31 that include both LBS and RBS.

TABLE 4
Plastid transformation of tobacco

| | | Leaf | | | |
|---|---|---|---|---|---|
| Vector^{a} | Enzyme^{b} | age^{c} | #Segments^{d} | #Calli^{e} | % Calli^{f} |
| no DNA | | young | 194 | 26 | 13 |
| no DNA | | old | 238 | 44 | 18 |
| pPRV111A | | young | 225 | 44 | 20 |
| pPRV111A | | old | 234 | 30 | 13 |
| LpPRV111A | EcoRV | young | 135 | 44 | 33 |
| LpPRV111A | EcoRV | old | 113 | 21 | 19 |
| TCpPRV111A | EcoRV/SacI | young | 29 | 15 | 52 |
| TCpPRV111A | EcoRV/SacI | old | 48 | 36 | 53 |

^{a}Data from total of seven experiments: particle bombardment with tungsten particles coated with either no DNA, the circular vector pPRV111A, or the linearized vectors LpPRV111A (four experiments) and TCpPRV111A (three experiments).
^{b}Restriction enzymes used for linearization of pPRV111A. The EcoRV digest was complete and yielded a single linear band, LpPRV111A. The EcoRV/SacI digest was complete with EcoRV but only partial with Sad, thus yielding three bands corresponding to LpPRV111A, TCpPRV111A, and residual pUC119.
^{c}Age of leaves used for bombardment.
^{d}Total number of leaf segments.
^{e}Number of sections with callus growth.
^{f}Percentage of leaf segments with callus growth.

TABLE 5
Efficiency of tobacco plastid transformation
using PCR for the aadA transgene

| | | Band Intensity^{c} | | | | |
|---|---|---|---|---|---|---|
| Tissue Type^{a} | #Positive/Total^{b} | − | +/− | + | ++ | +++ |
| no DNA-Young | 5/10 | 5 | 2 | 2 | 0 | 1 |
| no DNA-Old | 11/28 | 17 | 7 | 3 | 1 | 0 |
| pPRV111A-Young | 3/6 | 3 | 1 | 1 | 0 | 1 |
| pPRV1111A-Old | 10/26 | 14 | 0 | 10 | 2 | 0 |
| LpPRV111A-Young | 18/21 | 3 | 0 | 4 | 13 | 1 |
| LpPRV111A-Old | 21/28 | 7 | 3 | 13 | 5 | 0 |

^{a}Data from single experiment: biolistic bombardment with tungsten particles coated with either no DNA, the circular vector pPRV111A, or the linearized vector LpPRV111A (restriction digestion of pPRV111A with EcoRV/SacI).
^{b}Total tissue DNA (ttDNA) was prepared from leaf pieces after 3-4 weeks on selective medium. Number ttDNA samples with PCR product using aadA primers/total ttDNA samples tested.
^{c}Relative quantification of the amount of PCR product (visual inspection): − no product, +/− extremely faint DNA band, + to +++ increasing amounts of DNA.

TABLE 6
End sequences of maize ptDNA and putative tobacco and liverwort
ptDNA ends determined by sequence alignment to maize ptDNA ends

| | | | | Location | | Location | | Location |
|---|---|---|---|---|---|---|---|---|
| Maize ^{a} | Method^{b} | #Seq'ed ^{c} | Region ^{d} | (nt)^{e} | Tobacco | (nt)^{f} | Liverwort | (nt)^{f} |
| End1/5 | PCR/plasmid | 3/5 | IRb | 94976 | | 102583 | | 81931 |
| | | | IRa | 127767 | | 140047 | | 120189 |
| End2 | PCR | 3 | IRb | 94143 | | 101982 | SSC | none; |
| | | | | | | | | 97457 |
| | | | IRa | 128600 | | 140648 | | |
| End3 | PCR | 1 | IRb | 87402 | | none; | SSC | none; |
| | | | | | | 95104 | | 99780 |
| | | | IRa | 135341 | | none; | | |
| | | | | | | 147526 | | |
| End4 | PCR | 2 | IRb | 84555 | | 88371 | LSC | none; |
| | | | | | | | | 80541 |
| | | | IRa | 138188 | | 154259 | | |
| End6 | plasmid | 3 | IRb | 93863 | LSC | none; | LSC | 5318 |
| | | | | | | 57102 | | |
| | | | IRa | 128880 | | | | |

^{a} Ends identified for maize ptDNA. Sequence comparisons to identify the location of ends within the maize plastid genome were performed both independently for PCR products and plasmids and then as a combined set if similar end locations were found. Thus End1 (as determined with PCR products) and End5 (as determined with plasmids) were identified as the same end sequence by ClustalW alignment to the maize ptDNA sequence.
^{b}End determined by sequencing PCR product or plasmid with ptDNA insert. M13 primers were used for sequencing reactions.
^{c} Number of PCR products and/or plasmids sequenced (#Seq'ed) to determine each End. A total of five different ptDNA preparations were used for End sequencing.
^{d} Region of end sequences on tobacco and liverwort plastid genomes: LSC, long single copy region; SSC, short single copy region; IRb, inverted repeat b; IRa, inverted repeat a.
^{e}Nucleotide location corresponds to maize ptDNA sequence accession X86563.
^{f}Location of putative ends for tobacco and liverwort ptDNAs as compared to sequenced ends of maize ptDNA. Nucleotide location corresponds sequence accession NC_001879 and X04465, respectively. Comparisons were preformed with MacVector. None as determined by “Align to reference” (120 bp of Zm End sequences) which predicts >90% identity. For “ClustalW alignment”, there was 68% ID tobacco ptDNA to ZmEnd3 and 84% ID to ZmEnd6 and for liverwort 69% ID to ZmEnd2, 78% ID to ZmEnd3, and 63% ID to ZmEnd4.

TABLE 7
Location of tobacco and liverwort ptDNA end sequences

| | |
|---|---|
| Region^{a} | Location (nt)^{b} |

| | | | |
|---|---|---|---|
| Tobacco | LSC | End1 | 2369 |
| Restriction digest^{c} | LSC | End2 | 10893 |
| | LSC | End3 | 84436 |
| | IRb | End4 | 95913 |
| | IRa | | 147857 |
| | IRb | End5 | 107026 |
| | IRa | | 135861 |
| | SSC | End6 | 121313 |
| Vector^{d} | IRb | V-End7-LBS | 104087 |
| | IRa | | 138543 |
| | IRb | V-End7-RBS^{e} | 101190 |
| | IRa | | 141440 |
| | IRb | V-End8-RBS^{f} | 101675 |
| | IRa | | 140955 |
| Liverwort | IRb | V-End1-LBS^{h} | 83881 |
| Vector^{g} | IRa | | 118239 |
| | IRb | V-End1-RBS | 84883 |
| | IRa | | 117237 |

^{a}Region of end sequences on tobacco and liverwort plastid genomes: LSC, long single copy region; SSC, short single copy region; IRb, inverted repeat b; IRa, inverted repeat a.
^{b}Nucleotide (nt) on tobacco plastid genome of end sequences.
^{c}The end sequences were determined (±5000 bp) by restriction digestion (Scharff and Koop 2006 Plant Mol Biol 62: 611-621; Scharff and Koop 2007 Plant J 50: 782-794).
^{d}The exact end nucleotide for linearized vectors LpPRV111A and LpPRV131B if cut to give two fragments, cloning plasmid + LBS-transgene-RBS.
^{e}For vector TCpPRV111A-SacI, the end is at V-End7-LBS and is 1504 bp from the tobacco end predicted to be homologous to ZmEnd1/5 (Table 6).
^{f}For vector LpPRV111A-EcoRV, the end is at V-End8-RBS and is 307 bp from the tobacco end predicted to be homologous to ZmEnd2 (Table 6).
^{g}The exact end nucleotide for linearized vectors LpCS31 if cut to give two fragments, cloning plasmid + LBS-transgene-RBS.
^{h}For vectors LpCS31-SacI and TCpCS31-NotI/SacII, the end is at V-End1-RBS and is 2952 bp from the liverwort end predicted to be homologous to ZmEnd1/5. The end at V-End1-LBS is 1950 bp from the liverwort end predicted to be homologous to ZmEnd1/5 (Table 6).

TABLE 8
Plasmids/vectors

| | Size | |
|---|---|---|
| Plasmid/vector | (kb) | Insert Sequence |
| pzmcpGFP | 3.4 | gfp |
| pPrrnpsbA53 | 3.2 | Prrn5′psbA3′psbA |
| pZMCP112 | 4.5 | End5-5′ end IRb |
| | | LBS1-RBS1 |
| pZMCP113 | 4.2 | End5-3′ end IRb |
| | | LBS2-RBS2 |
| pZMCP120 | 3.9 | Prrn5′psbAgfp3′psbA |
| pZMCP150 | 5.7 | LBS1-Prrn5′psbAgfp3′psbA-RBS1 |
| pZMCP152 | 5.5 | LBS2-Prrn5′psbAgfp3′psbA-RBS2 |

TABLE 9
Maize tissues with GFP fluorescence imaged with a hand-
held laser and dot blot hybridization with a gfp probe

| | Mature embryos^{b} | | | | Dark-grown stalk^{c} | | |
|---|---|---|---|---|---|---|---|
| | | #GFP/ | | | | #GFP/ | |
| Vector^{a} | #Plates | total^{d} | % GFP^{e} | gfp-hyb^{f} | #Plates | total^{d} | % GFP^{e} |
| No DNA | 4 | 2/54 | 4 | 0/8 | 1 | 0/17 | 0 |
| pZMCP150 | 7 | 22/91 | 24 | 2/16 | nd | | |
| TCpZMCP150 | 7 | 26/92 | 28 | 2/16 | nd | | |
| pZMCP152 | 5 | 13/81 | 17 | 3/16 | 3 | 6/50 | 12 |
| TCpZMCP152 | 5 | 16/75 | 21 | 3/16 | 2 | 3/31 | 10 |
| TC2pZMCP152 | 1 | 7/15 | 47 | | | | |
| TC3pZMCP152 | 1 | 6/15 | 40 | | | | |

^{a}Vector used for bombardment for each plate.
^{b}Data from 3-4 biolistics experiments, except single experiment for TC2pZMCP152 and TC3pZMCP152. Each plate contained 12-17 pieces of mature embryo.
^{c}Data from a single biolistics experiment. Each plate contained 14-17 pieces of stalk from dark-grown seedlings, nd: not done.
^{d}Number of pieces (embryo or stalk) with GFP sectors/total number of pieces of tissue. Visual inspection was performed with a dissecting microscope.
^{e}Percentage of calli/shoots sectors with GFP fluorescence.
^{f}Data from a single biolistics experiment. ttDNA was prepared from several pieces of tissue following biolistics, spotted onto N⁺ nylon membrane and hybridized with DIG-gfp probe. The number positive for gfp-hybridization/total number ttDNA samples is given.

TABLE 10
Maize cells with GFP-plastids

| | | | GFP | % Cells with |
|---|---|---|---|---|
| Plate#^{a} | Vector^{b} | Tissue^{c} | by laser^{d} | GFP-plastids^{e} |
| T11 | None | Callus w/ shoot | − | 0 |
| T12 | pZMCP150 | Callus w/ shoot | + | ≦10 |
| T13 | pZMCP150 | Callus w/ shoot | − | 0 |
| | | Shoot | + | ≦15 |
| T14 | TCpZMCP150 | Callus | − | ≧75 |
| T16 | pZMCP152 | Callus | + | ≦25 |
| T17 | pZMCP152 | Callus | + | ≧50 |
| T18 | TCpZMCP152 | Callus | + | ≦25 |
| T19 | TCpZMCP152 | Callus | + | ≧50 |
| | | Shoot | + | 0 |

^{a}Single biolistics experiment. Each plate contained 12-17 pieces of mature embryo.
^{b}Vector used to bombard each plate.
^{c}A single piece (except for T13 and T19) of maize tissue was selected from each plate and examined microscopically for cells with GFP expression in plastids. The tissue was squashed onto a microscope slide. After microscopic examination, the tissue was scraped from the slide for preparation of ttDNA and PCR analysis.
^{d}All embryo pieces on each plate were assessed for GFP expression using a hand-held laser and dissecting microscope (Table 8). The single piece of tissue from each plate that was selected for analysis of GFP-plastids by epifluorescence microscopy was either positive (+) or negative (−) using this procedure.
^{e}Percentage of cells with GFP expression in plastids.

TABLE 11
Maize protoplasts with GFP-plastids

Maize Protoplasts^{b}

| Vector^{a} | #GFP (%) | #no GFP (%) |
|---|---|---|
| NoDNA | 0 | 26 (100%) |
| PZMCP152 | 15 (23%) | 50 (77%) |
| TC1pZMCP152 | 30 (51%) | 29 (49%) |
| TC4pZMCP152 | 22 (29%) | 55 (71%) |

^{a}Vectors used for bombardment for each plate. pZMCP152 is a circular vector. For linear vector TC1pZMCP152, the 3′ end of End5 is exposed at the LBS region. For linear vector TC4pZMCP152, the 3′end of End5 is “blocked” by a segment of the cloning plasmid at the LBS region.
^{b}Protoplasts were prepared from a single plate of transformed maize tissue, then examined for GFP expression in the plastids using fluorescence microscopy. White light was first used to identify individual protoplasts, then a GFP-filter was used to assess protoplasts with (#GFP) and without (#no GFP) GFP signal.

TABLE 12
Wheat tissues with dot blot hybridization with a gfp probe and
rice tissues with GFP expression imaged with a hand-held laser

| | Wheat^{b} | Rice^{c} |
|---|---|---|
| Vector^{a} | gfp-hyb | GFP |
| No DNA | 0/12 | 0/18 |
| pZMCP150 | 6/20 | nd |
| TCpZMCP150 | 6/20 | nd |
| PZMCP152 | 6/16 | 1/18 |
| TCpZMCP152 | 9/20 | 6/18 |
| TC2pZMCP152 | nd | 10/18 |
| TC3pZMCP152 | nd | 5/18 |

^{a}Vectors used to bombard each plate.
^{b}Two biolistics experiments were performed for wheat and a total of three plates of seeds bombarded with each vector and two with no DNA. Each plate contained 20 wheat seeds of which 4 to 8 seedlings from each plate were selected randomly after biolistics for analysis by blot hybridization. ttDNA was prepared from seedlings following biolistics, spotted onto N⁺ nylon membrane and hybridized with DIG-gfp probe. The number positive for gfp-hybridization/total number ttDNA samples is given, nd: not done.
^{c}Single biolistic experiment was performed. Each plate contained 18 rice seeds that were split into two pieces. Number of pieces tissue with GFP expression/total number of seeds was determined by viewing with hand-held laser and dissecting microscope.

In the description provided herein, any ranges provided herein include all the values in the ranges. It should also be noted that the term “or” is generally employed in its sense including “and/or” (i.e., to mean either one, both, or any combination thereof of the alternatives) unless the content clearly dictates otherwise. Also, as used in this specification and the appended claims, the singular forms “a,” “an,” and “the” include plural referents unless the content clearly dictates otherwise.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of plastid transformation, comprising: (a) introducing a linear DNA vector into a plastid of a plant tissue, wherein
(i) the plastid comprises substantially non-degraded plastid DNA, and
(ii) the linear DNA vector comprises
(1) a plastid DNA targeting sequence, and
(2) a transgene of interest.

2. The method of claim 1, wherein the plant is a monocot or a dicot.

3. The method of claim 1, wherein the plant is a liverwort or related species.

4. The method of claim 1, wherein the transgene of interest is selected from the group consisting of genes encoding therapeutic or prophylactic polypeptides, genes that provide or enhance herbicide resistance, insect resistance, fungal resistance, bacterial resistance, and stress tolerance, and genes that improve nitrogen fixation, mineral nutrition, plant yield, starch accumulation, fatty acid accumulation, protein accumulation, and photosynthesis.

5. The method of claim 1, wherein the linear DNA vector further comprises a gene encoding a selection marker.

6. The method of claim 5, wherein the selection marker is a gene providing resistance against spectinomycin, streptomycin, kanamycin, hygromycin, chloramphenicol, glyphosate or bialaphos.

7. The method of claim 5, wherein the selection marker is a marker for metabolic selection.

8. The method of claim 5, wherein the selection marker is a gene encoding a fluorescent protein.

9. The method of claim 1, wherein the plastid DNA targeting sequence comprises a terminal sequence of a plastid chromosomal DNA molecule.

10. The method of claim 9, wherein the plastid terminal sequence is at least 90% identical to a portion of SEQ ID NO: 1, 8, 15, 21, 29, 35, 41, or 47, which portion is at least 30 nucleotides in length.

11. The method of claim 9, wherein the plastid terminal sequence comprises:
at least 30 consecutive nucleotides of SEQ ID NO:2, 9, 16, 22, 27, 28, 30, 36, 42, 48, 53, 54, 55, 56, or 57 when the plant tissue is a maize tissue,
at least 30 consecutive nucleotides of SEQ ID NO:3, 10, 17, 23, 31, 37, 43, or 49 when the plant tissue is a wheat tissue,
at least 30 consecutive nucleotides of SEQ ID NO:4, 5, 11, 12, 18, 19, 24, 25, 32, 33, 38, 39, 44, 45, 50, or 51 when the plant tissue is a rice tissue,
at least 30 consecutive nucleotides of SEQ ID NO:6, 13, 20, 26, 34, 40, or 52 when the plant tissue is a tobacco tissue, or
at least 30 consecutive nucleotides of SEQ ID NO:7 or 46 when the plant tissue is a liverwort tissue.

12. The method of claim 1, wherein the linear DNA vector comprises a single-stranded overhang at either the 5′ end or the 3′ end, a single-stranded loop that may or may not covalently join the two DNA strands of the linear DNA vector, or a molecule that is not a nucleotide covalently joined to either the 5′ end or the 3′ end.

13. The method of claim 1, wherein the plant tissue is a non-green tissue.

14. The method of claim 13, wherein the non-green tissue is a portion of a mature embryo, a portion of a dark-grown seedling, a seed, or a portion of a seed.

15. The method of claim 1, wherein the plastid is a proplastid, etioplast, or other non-green plastid.

16. The method of claim 1, wherein step (a) is performed via biolistic bombardment of the plant tissue with microparticles coated with the linear DNA vector.

17. The method of claim 1, further comprising (b) culturing the plant tissue from step (a) without light.

18. The method of claim 1, further comprising (c) regenerating a transplastomic plant from the plant tissue from step (a) or step (b).

19. The method of claim 18, wherein the transplastomic plant is homoplasmic.

20. A transplastomic plant or a plant part obtained by the method of claim 1.

21. A progeny of a plant or plant part of claim 20.

22. A linear DNA vector for plastid transformation in a plant, comprising:
(1) a plastid DNA targeting sequence that comprises a plastid terminal sequence, and
(2) an expression cassette that comprises:
(a) optionally a promoter active in the plastids of the plant to be transformed,
(b) a DNA insertion site for receiving a transgene of interest,
(c) optionally one or more selection markers, and
(d) optionally a DNA sequence encoding a transcription termination region active in the plastids of the plant to be transformed.

23. The linear DNA vector of claim 22, further comprising a transgene of interest inserted at the DNA insertion site.

24. The method of claim 7, wherein the selection marker is a gene providing metabolism of mannose.
